(19) United States
(12) Patent Application Publication
(10) Pub. No.: US 2023/0416752 A1
(43) Pub. Date: Dec. 28, 2023

(54) CHEMICAL MODIFICATIONS FOR INHIBITING EXPRESSION OF ALDH2

(71) Applicant: Dicerna Pharmaceuticals, Inc., Lexington, MA (US)

(72) Inventors: Bob Dale BROWN, Littleton, MA (US); Henryk T. DUDEK, Belmont, MA (US); Utsav SAXENA, Watertown, MA (US)

(21) Appl. No.: 18/036,801

(22) PCT Filed: Nov. 12, 2021
(86) PCT No.: PCT/US2021/072370

§ 371 Date: May 12, 2023

Related U.S. Application Data
(60) Provisional application No. 63/113,689, filed on Nov. 13, 2020.

(51) Int. Cl. C12N 15/113; A61K 31/145; A61K 31/713

(52) U.S. Cl. CPC C12N 15/1137; C12Y 102/01003; A61K 31/145; A61K 31/713; C12N 2310/14; C12N 2310/321; C12N 2310/315; C12N 2310/3125; C12N 2320/32; C12N 2320/53

(57) ABSTRACT

This disclosure relates to chemically modified oligonucleotides, compositions, and methods useful for reducing ALDH2 expression, to treat alcoholism. Disclosed oligonucleotide for the reduction of ALDH2 expression is modified for enhanced pharmacological properties.

### TECHNICAL FIELD

The present application relates to chemically modified oligonucleotides and use thereof for the treatment of alcoholism and associated conditions.

### REFERENCE TO SEQUENCE LISTING

A Sequence Listing is submitted concurrently with the specification as an ASCII formatted text file, with a file name of DRNA074_ST25.txt, a creation date of Nov. 13, 2020, and a size of 19 kilobytes. The information in the electronic format of the Sequence Listing is part of the specification and is hereby incorporated herein by reference in its entirety.

### BACKGROUND

Alcoholism may be classified as alcohol abuse, alcohol use disorder or alcohol dependence. Alcohol use disorder (AUD) represents a highly prevalent, costly, and often untreated condition in the United States and globally. Pharmacotherapy offers a promising avenue for treating AUD and for improving clinical outcomes for this debilitating disorder. There is a great need for developing novel medications to treat AUD. The present disclosure presents chemically modified oligonucleotides for treating AUD through aldehyde dehydrogenase 2 (ALDH2) inhibition.

### SUMMARY OF THE INVENTION

The present invention is based, at least in part, upon the development of potent oligonucleotides producing durable RNAi-based ALDH2 inhibitors. Certain aspects of the disclosure relate to the chemical modifications of the oligonucleotides and related methods for treating alcoholism in a subject.

Existing pharmaceutical approaches to treat AUD include naltrexone, acamprosate, and disulfiram an aldehyde dehydrogenase-2 (ALDH2) inhibitor. ALDH2 is a conserved detoxifying mitochondrial enzyme, notably implicated in the metabolism of aldehydes. The systemic metabolism of ethanol (“alcohol”) is initiated with the first metabolism to acetaldehyde by alcohol dehydrogenase (ADH), then into acetate by ALDH2. Additionally, ALDH2 plays a key role in oxidizing lipid peroxidation products generated under oxidative stress, such as 4-hydroxy-2-nonenal and malondialdehyde. An estimated 8% of the world population, mainly of East Asian descent, harbor the ALDH2*2 allele which encodes for a nonfunctioning ALDH2 enzyme, resulting in acetaldehyde buildup in the blood and organs, such as liver and brain, after alcohol consumption. In these individuals, acetaldehyde accumulation causes facial flushing, and unpleasant feelings such as nausea, headaches, cardiac palpitations, and overall discomfort. Due to this difficulty in metabolism ALDH2-deficient individuals are at lower risks of developing AUD. For these reasons, approaches that aim to specifically and reversibly inhibit ALDH2 activity would be of great interest in the treatment of AUD.

In certain embodiments, the chemical modifications of the oligonucleotides of the present disclosure provide surprisingly enhanced chemically stability and reduced the cost of manufacturing. In certain embodiments, the chemical modifications include reducing fluorine content (see, e.g., PCT/US20/53999, Weimin Wang et al, which is incorporated herein by reference in their entirety). In certain embodiments, the reduced fluorine content increases the yield in the manufacturing thereby significantly lowering costs. In certain embodiments, reduction in fluorine content decreases the defluorination impurity. In some embodiments, potent and stable RNAi oligonucleotides are useful for reducing ALDH2 activity, and thereby decreasing alcohol tolerance and/or the desire to consume alcohol. In some embodiments, RNAi oligonucleotides disclosed herein have, among other characteristics, retained potency, retained, or increased duration of action, retained high therapeutic index, improved stability, improved bioavailability, improved targeting, eased manufacturing, lower toxicity and/or other improved pharmacological properties as compared to prior oligonucleotides.

The primary enzymes involved in alcohol metabolism are alcohol dehydrogenase (ADH) and aldehyde dehydrogenase (ALDH). Both enzymes occur in several forms that are encoded by different genes; moreover, there are variants (i.e., alleles) of some of these genes that encode enzymes with different characteristics, and which have different population distributions by ethnic group. Which ADH or ALDH alleles a person carries influence his or her level of alcohol consumption and risk of alcoholism. Researchers to date primarily have studied coding variants in the ADH1B, ADH1C, and ALDH2 genes that are associated with altered kinetic properties of the resulting enzymes.

One aspect of the present disclosure provides an oligonucleotide for reducing expression of ALDH2, the oligonucleotide comprising an antisense strand having a sequence from 5′ to 3′ set forth as UAAACUGAGUUUCAUCCACCGG (SEQ ID NO: 1) and a sense strand having a sequence from 5′ to 3′ set forth as

(SEQ ID NO: 2)
GGUGGAUGAAACUCAGUUUAGCAGCCGAAAGGCUGC.

In some embodiments, the oligonucleotide comprises at least one modified nucleotide. In some embodiments, all the nucleotides of the oligonucleotide are modified. In some embodiments, the modified nucleotide comprises a 2′-modification. In some embodiments, the 2′-modification is a 2′-fluoro or 2′-O-methyl. In some embodiments, one or more of the following positions are modified with a 2′-O-methyl: positions 1-7 and 12-36 of the sense strand and/or positions 1, 6, 8-13 and 15-22 of the antisense strand. In some embodiments, all of positions 1-7 and 12-36 of the sense strand and positions 1, 6, 8-13 and 15-22 of the antisense strand are modified with a 2′-O-methyl. In some embodiments, one or more of the following positions are modified with a 2′-fluoro: positions 8-11 of the sense strand and/or positions 2-5, 7 and 14 of the antisense strand. In some embodiments, all of positions 8-11 of the sense strand and positions 2-5, 7 and 14 of the antisense strand are modified with a 2′-fluoro.

In some embodiments, the oligonucleotide comprises at least one modified internucleotide linkage. In some embodiments, the at least one modified internucleotide linkage is a phosphorothioate linkage. In some embodiments, the oligonucleotide has a phosphorothioate linkage between one or more of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

In some embodiments, the uridine at the first position of the antisense strand comprises a phosphate analog.

In some embodiments, the oligonucleotide comprises the following structure at position 1 of the antisense strand:

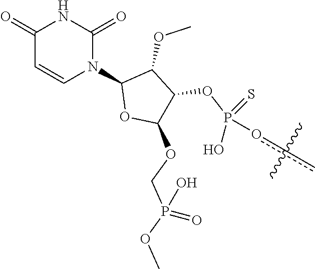

In some embodiments, one or more of the nucleotides of the -GAAA- sequence on the sense strand is conjugated to a monovalent GalNAc moiety. In some embodiments, each of the A nucleotides of the -GAAA- sequence on the sense strand is conjugated to a monovalent GalNAc moiety. In some embodiments, the ademA GalNAc represents the structure:

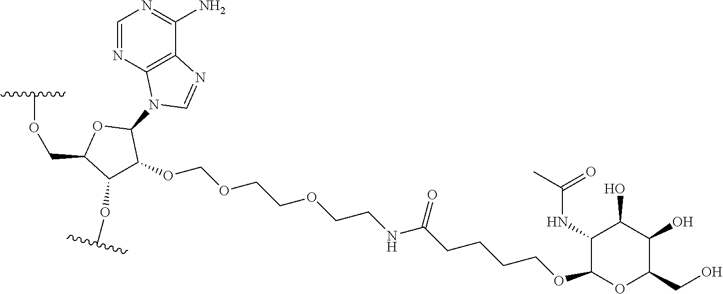 [SMILES: *OC[C@H]1O[C@@H](n2cnc3c(N)ncnc32)[C@H](OCOCCOCCNC(=O)CCCCO[C@@H]2O[C@H](CO)[C@H](O)[C@H](O)[C@H]2NC(C)=O)[C@@H]1O*]

In some embodiments, the oligonucleotide for reducing expression of ALDH2 comprises an antisense strand having a sequence from 5′ to 3′ set forth as UAAACUGAGUUUCAUCCACCGG (SEQ ID NO: 1) and a sense strand having a sequence from 5′ to 3′ set forth as GGUGGAUGAAACUCAGUUUAGCAGCCGAAAGGCUGC (SEQ ID NO: 2),
wherein all of positions 1-7 and 12-36 of the sense strand and positions 1, 6, 8-13 and 15-22 of the antisense strand are modified with a 2′-O-methyl, and all of positions 8-11 of the sense strand and positions 2-5, 7 and 14 of the antisense strand are modified with a 2′-fluoro;
wherein the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand;
wherein the oligonucleotide comprises the following structure at position 1 of the antisense strand:

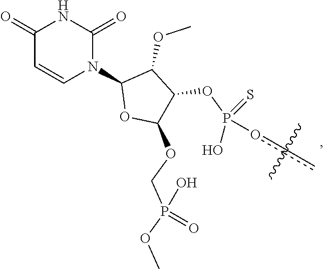

and
wherein each of the Adenosine (A) nucleotides of the -GAAA- sequence on the sense strand is conjugated to a monovalent GalNAc moiety comprising the structure:

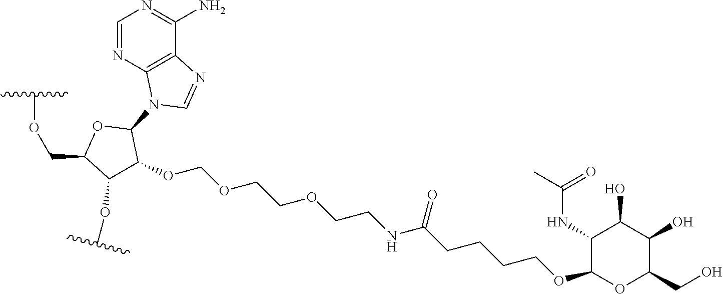 [SMILES: *OC[C@H]1O[C@@H](n2cnc3c(N)ncnc32)[C@H](OCOCCOCCNC(=O)CCCCO[C@@H]2O[C@H](CO)[C@H](O)[C@H](O)[C@H]2NC(C)=O)[C@@H]1O*]

Other aspects of the present disclosure provide a composition comprising any of the oligonucleotides described herein and Na+ counterions.

A composition having the chemical structure as depicted in FIG. 3 is also provided.

Another aspect of the present disclosure provides a method comprising administering a composition of the present disclosure to a subject. In some embodiments, the method results in a decreased ethanol tolerance in a subject. In some embodiments, the method results in an inhibition of ethanol intake by a subject. In some embodiments, the method results in a decreased desire of a subject to consume ethanol. In some embodiments, the subject to be treated suffers from alcoholism.

### BRIEF DESCRIPTION OF THE DRAWINGS

The accompanying drawings, which are incorporated in and constitute a part of this specification, illustrate certain embodiments, and together with the written description, serve to provide non-limiting examples of certain aspects of the compositions and methods disclosed herein.

FIG. 1 is a graph showing impact of 2′-OMe substitution on in vivo activity evaluation of GalNAc-conjugated ALDH2 oligonucleotides. Oligonucleotides were subcutaneously administered to mice at 0.5 mg/kg. The data show the amount of ALDH2 mRNA remaining at day 4 following administration normalized to PBS control.

FIG. 2 is a graph showing the results of a duration study of GalNAc-conjugated ALDH2 oligonucleotides with different modification patterns in non-human primates (NHP). A single dose (3 mg/kg) of the oligonucleotides was subcutaneously administered to non-human primates. The data show the amount of ALDH2 mRNA remaining 4-, 8-, 12-, and 16-weeks following administration, relative to the amount of ALDH2 mRNA prior to administration.

FIG. 3 is a schematic depicting the structure and chemical modification patterns of the disclosed oligonucleotide.

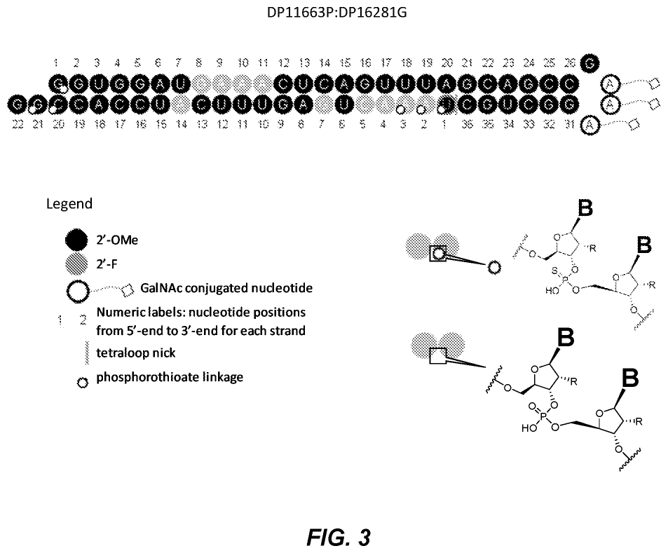

### DETAILED DESCRIPTION OF THE INVENTION

Aspects of the present disclosure provide an oligonucleotide (e.g., RNA interference oligonucleotide) comprising chemical modification patterns for reducing ALDH2 expression in cells with better potency and durability, particularly liver cells (e.g., hepatocytes) for the treatment of alcoholism. Accordingly, in related aspects, the disclosure provides methods of treating alcoholism that involve selectively reducing ALDH2 gene expression in liver. In certain embodiments, ALDH2 targeting oligonucleotides provided herein are designed for delivery to selected cells of target tissues (e.g., liver hepatocytes) to treat alcoholism in a subject, where the oligonucleotides have increased resistance to degradation and/or display increased duration in the selected cells.

The effects of ingested beverage alcohol (i.e., ethanol) on different organs, including the brain, depend on the ethanol concentration achieved and the duration of exposure. Both variables, in turn, are affected by the absorption of ethanol into the blood stream and tissues as well as by ethanol metabolism (Hurley et al., “The pharmacogenomics of alcoholism,” In: Pharmacogenomics: The Search for Individualized Therapies., Weinheim, Germany: Wiley-VCH, pp. 417-441 (2002)). The main site of ethanol metabolism is the liver, although some metabolism also occurs in other tissues and can cause local damage there. The main pathway of ethanol metabolism involves its conversion (i.e., oxidation) to acetaldehyde, a reaction that is mediated (i.e., catalyzed) by enzymes known as alcohol dehydrogenases. According to the current invention, the inhibition of expression of one or more of these alcohol dehydrogenases, preferably ALDH2, prevents or eliminates the ability of a subject to breakdown alcohol-leading to a prevention in the ‘high’ associated with ingesting it in humans.

Aldehyde dehydrogenase 2 (ALDH2), a key enzyme for detoxification the ethanol metabolite acetaldehyde and has been recognized as a potential therapeutic target to treat alcohol use disorders (AUDs). Disulfiram, a potent ALDH2 inhibitor, is an approved drug for the treatment of AUD but has clinical limitations due to its side effects. In terms of organ system contribution, it is known that the liver is the major organ responsible for acetaldehyde metabolism, a cumulative effect of ALDH2 from other organs likely also contributes to systemic acetaldehyde clearance. According to the present invention, liver-targeted knockdown of ALDH2 expression via siRNA can decrease alcohol preference and can be the basis for the treatment of AUD.

In certain aspects, the present disclosure provides ALDH2 targeting oligonucleotides with increased yield and lower impurity during manufacturing.

In further aspects, the ALDH2 targeting oligonucleotides have decreased fluorine content. In some aspects, the fluorine content of pyrimidine bases is decreased.

Further aspects of the disclosure, including a description of defined terms, are provided below.

### I. Definitions

Alcoholism: As used herein, the term, “alcoholism” refers to repeated use of ethanol by an individual despite recurrent adverse consequences, which may or may not be combined with tolerance, withdrawal, and/or an uncontrollable drive to consume alcohol. Alcoholism may be classified as alcohol abuse, alcohol use disorder or alcohol dependence. A variety of approaches may be used to identify an individual suffering from alcoholism. For example, the World Health Organization has established the Alcohol Use Disorders Identification Test (AUDIT) as a tool for identifying potential alcohol misuse, including dependence and other similar tests have been developed, including the Michigan Alcohol Screening Test (MAST). Laboratory tests may be used to evaluate blood markers for detecting chronic use and/or relapse in alcohol drinking, including tests to detect levels of gamma-glutamyl transferase (GGT), mean corpuscular volume (red blood cell size), aspartate aminotransferase (AST), alanine aminotransferase (ALT), carbohydrate-deficient transferring (CDT), ethyl glucuronide (EtG), ethyl sulfate (EtS), and/or phosphatidylethanol (PEth). Animal models (e.g., mouse models) of alcoholism have been established (see, e.g., Rijk et al., “A mouse model of alcoholism,” PHYSIOL BEHAV., 1982, 29(5):833-39; Elizabeth Brandon-Warner, et al., “Rodent Models of Alcoholic Liver Disease: Of Mice and Men,” ALCOHOL, 2012; 46(8):715-25; and Adeline Bertola, et al., “Mouse model of chronic and binge ethanol feeding (the NIAAA model),” NATURE PROTOCOLS, 2013, 8:627-37).

ALDH2: As used herein, the term, “ALDH2” refers to the aldehyde dehydrogenase 2 family (mitochondrial) gene. ALDH2 encodes proteins that belong to the aldehyde dehydrogenase enzyme family and that function as the second enzyme of the oxidative pathway of alcohol metabolism that synthesizes acetate (acetic acid) from ethanol. Homologs of ALDH2 are conserved across a range of species, including human, mouse, rat, non-human primate species, and others (see, e.g., NCBI HOMOLOGENE: 55480). ALDH2 also has homology with other aldehyde dehydrogenase encoding genes, including, for example, ALDH1A1. In humans, ALDH2 encodes at least two transcripts, namely NM 000690.3 (variant 1) and NM_001204889.1 (variant 2), each encoding a different isoform, NP_000681.2 (isoform 1) and NP_001191818.1 (isoform 2), respectively. Transcript variant 2 lacks an in-frame exon in the 5′ coding region, compared to transcript variant 1, and encodes a shorter isoform (2), compared to isoform 1. Polymorphisms in ALDH2 have been identified (see, e.g., Chang et al., “ALDH2 polymorphism and alcohol-related cancers in Asians: a public health perspective,” J BIOMED SCI., 2017, 24(1):19).

Approximately: As used herein, the term “approximately” or “about,” as applied to one or more values of interest, refers to a value that is like a stated reference value. In certain embodiments, the term “approximately” or “about” refers to a range of values that fall within 25%, 20%, 19%, 18%, 17%, 0%, 19% 18%6, 15%, 14%, 3%, 12%, %11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Administering: As used herein, the terms “administering” or “administration” means to provide a substance (e.g., an oligonucleotide) to a subject in a manner that is pharmacologically useful (e.g., to treat a condition in the subject).

Asialoglycoprotein receptor (ASGPR): As used herein, the term “Asialoglycoprotein receptor” or “ASGPR” refers to a bipartite C-type lectin formed by a major 48 kDa (ASGPR-1) and minor 40 kDa subunit (ASGPR-2). ASGPR is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins).

Combination: As used herein, “combination product”, “combination therapy”, “polytherapy” and the like refer to a therapy used for the treatment of a disease or disorder using more than one therapeutic agent or more than one medicament or modality. The therapeutic agents comprising a combination product may be dosed concurrently, intermittently or in any sequence. A combination product may comprise, for example, two oligonucleotides or an oligonucleotide combined with an antibody or small-molecule drug. For such therapies, the dosages of each agent used may vary to optimize and/or enhance patient outcome.

Complementary: As used herein, the term “complementary” refers to a structural relationship between nucleotides (e.g., two nucleotides on opposing nucleic acids or on opposing regions of a single nucleic acid strand) that permits the nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. In some embodiments, two nucleic acids may have nucleotide sequences that are complementary to each other to form regions of complementarity, as described herein.

Deoxyribonucleotide: As used herein, the term “deoxyribonucleotide” refers to a nucleotide having a hydrogen at the 2′ position of its pentose sugar as compared with a ribonucleotide. A modified deoxyribonucleotide is a deoxyribonucleotide having one or more modifications or substitutions of atoms other than at the 2′ position, including modifications or substitutions in or of the sugar, phosphate group or base.

Double-stranded oligonucleotide: As used herein, the term “double-stranded oligonucleotide” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base-pairing of duplex region(s) of a double-stranded oligonucleotide is formed from a single nucleic acid strand that is folded (e.g., via a hairpin) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

Duplex: As used herein, the term “duplex,” in reference to nucleic acids (e.g., oligonucleotides), refers to a structure formed through complementary base-pairing of two antiparallel sequences of nucleotides.

Excipient: As used herein, the term “excipient” refers to a non-therapeutic agent that may be included in a composition, for example, to provide or contribute to a desired consistency or stabilizing effect.

Hepatocyte: As used herein, the term “hepatocyte” or “hepatocytes” refers to cells of the parenchymal tissues of the liver. These cells make up approximately 70-85% of the liver's mass and manufacture serum albumin, fibrinogen, and the prothrombin group of clotting factors (except for Factors 3 and 4). Markers for hepatocyte lineage cells may include but are not limited to: transthyretin (Ttr), glutamine synthetase (Glul), hepatocyte nuclear factor 1a (Hnf1a), and hepatocyte nuclear factor 4a (Hnf4a). Markers for mature hepatocytes may include but are not limited to: cytochrome P450 (Cyp3a11), fumarylacetoacetate hydrolase (Fah), glucose 6-phosphate (G6p), albumin (Alb), and OC2-2F8. See, e.g., Huch et al., NATURE, 2013, 494(7436):247-50, the contents of which relating to hepatocyte markers is incorporated herein by reference.

Loop: As used herein, the term “loop” refers to an unpaired region of a nucleic acid (e.g., oligonucleotide) that is flanked by two antiparallel regions of the nucleic acid that are sufficiently complementary to one another, such that under appropriate hybridization conditions (e.g., in a phosphate buffer, in a cells), the two antiparallel regions, which flank the unpaired region, hybridize to form a duplex (referred to as a “stem”).

Modified Internucleotide Linkage: As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

Modified Nucleotide: As used herein, the term “modified nucleotide” refers to a nucleotide having one or more chemical modifications compared with a corresponding reference nucleotide selected from: adenine ribonucleotide, guanine ribonucleotide, cytosine ribonucleotide, uracil ribonucleotide, adenine deoxyribonucleotide, guanine deoxyribonucleotide, cytosine deoxyribonucleotide and thymidine deoxyribonucleotide. In some embodiments, a modified nucleotide is a non-naturally occurring nucleotide. In some embodiments, a modified nucleotide has one or more chemical modifications in its sugar, nucleobase and/or phosphate group. In some embodiments, a modified nucleotide has one or more chemical moieties conjugated to a corresponding reference nucleotide. Typically, a modified nucleotide confers one or more desirable properties to a nucleic acid in which the modified nucleotide is present. For example, a modified nucleotide may improve thermal stability, resistance to degradation, nuclease resistance, solubility, bioavailability, bioactivity, reduced immunogenicity, etc. In certain embodiments, a modified nucleotide comprises a 2′-O-methyl or a 2′-F substitution at the 2′ position of the ribose ring.

Nicked Tetraloop Structure: A “nicked tetraloop structure” is a structure of a RNAi oligonucleotide characterized by the presence of separate sense (passenger) and antisense (guide) strands, in which the sense strand has a region of complementarity to the antisense strand such that the two strands form a duplex, and in which at least one of the strands, generally the sense strand, extends from the duplex in which the extension contains a tetraloop and two self-complementary sequences forming a stem region adjacent to the tetraloop, in which the tetraloop is configured to stabilize the adjacent stem region formed by the self-complementary sequences of the at least one strand.

Oligonucleotide: As used herein, the term “oligonucleotide” refers to a short nucleic acid, e.g., of less than 100 nucleotides in length. An oligonucleotide can comprise ribonucleotides, deoxyribonucleotides, and/or modified nucleotides including, for example, modified ribonucleotides. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

Overhang: As used herein, the term “overhang” refers to terminal non-base-pairing nucleotide(s) resulting from one strand or region extending beyond the terminus of a complementary strand with which the one strand or region forms a duplex. In some embodiments, an overhang comprises one or more unpaired nucleotides extending from a duplex region at the 5′ terminus or 3′ terminus of a double-stranded oligonucleotide. In certain embodiments, the overhang is a 3′ or 5′ overhang on the antisense strand or sense strand of a double-stranded oligonucleotide.

Pharmaceutically acceptable: As used herein, the term “pharmaceutically acceptable” refers to compounds or compositions which are generally safe, non-toxic and neither biologically nor otherwise undesirable, and includes a compound or composition that is acceptable for human pharmaceutical and veterinary use. The compound or composition may be approved or approvable by a regulatory agency or listed in the U.S. Pharmacopeia or other generally recognized pharmacopeia for use in animals, including humans.

Pharmaceutically acceptable salts: As used herein, the term “pharmaceutically acceptable salts” refers to physiologically and pharmaceutically acceptable salts of the compounds of the invention: i.e., salts that retain the desired biological activity of the parent oligonucleotides and do not impart undesired toxicological effects thereto.

Pharmaceutically acceptable excipient, carrier or adjuvant: As used herein, the term “pharmaceutically acceptable excipient, carrier or adjuvant” refers to an excipient, carrier or adjuvant that can be administered to a subject, together with at least one therapeutic agent (e.g., an oligonucleotide of the present disclosure), and which does not destroy the pharmacological activity thereof and is generally safe, nontoxic and neither biologically nor otherwise undesirable when administered in doses sufficient to deliver a therapeutic amount of the agent.

Phosphate analog: As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, e.g., International Patent Application PCT/US2017/049909, filed on Sep. 1, 2017, U.S. Provisional Application No. 62/383,207, filed on Sep. 2, 2016, and 62/393,401, filed on Sep. 12, 2016, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al., NUCLEIC ACIDS RES., 2015, 43(6):2993-3011, the contents of each of which relating to phosphate analogs are incorporated herein by reference).

Reduced expression: As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a double-stranded oligonucleotide (e.g., one having an antisense strand that is complementary to ALDH2 mRNA sequence) may result in a decrease in the amount of RNA transcript, protein and/or enzymatic activity (e.g., encoded by the ALDH2 gene) compared to a cell that is not treated with the double-stranded oligonucleotide. Similarly, “reducing expression” as used herein refers to an act that results in reduced expression of a gene (e.g., ALDH2).

Region of Complementarity: As used herein, the term “region of complementarity” refers to a sequence of nucleotides of a nucleic acid (e.g., a double-stranded oligonucleotide) that is sufficiently complementary to an antiparallel sequence of nucleotides (e.g., a target nucleotide sequence within an mRNA) to permit hybridization between the two sequences of nucleotides under appropriate hybridization conditions, e.g., in a phosphate buffer, in a cell, etc. A region of complementarity may be fully complementary to a nucleotide sequence (e.g., a target nucleotide sequence present within an mRNA or portion thereof). For example, a region of complementary that is fully complementary to a nucleotide sequence present in an mRNA has a contiguous sequence of nucleotides that is complementary, without any mismatches or gaps, to a corresponding sequence in the mRNA. Alternatively, a region of complementarity may be partially complementary to a nucleotide sequence (e.g., a nucleotide sequence present in an mRNA or portion thereof). For example, a region of complementary that is partially complementary to a nucleotide sequence present in an mRNA has a contiguous sequence of nucleotides that is complementary to a corresponding sequence in the mRNA but that contains one or more mismatches or gaps (e.g., 1, 2, 3, or more mismatches or gaps) compared with the corresponding sequence in the mRNA, provided that the region of complementarity remains capable of hybridizing with the mRNA under appropriate hybridization conditions.

Ribonucleotide: As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

RNAi Oligonucleotide: As used herein, the term “RNAi oligonucleotide” refers to either (a) a double stranded oligonucleotide having a sense strand (passenger) and antisense strand (guide), in which the antisense strand or part of the antisense strand is used by the Argonaute 2 (Ago2) endonuclease in the cleavage of a target mRNA or (b) a single stranded oligonucleotide having a single antisense strand, where that antisense strand (or part of that antisense strand) is used by the Ago2 endonuclease in the cleavage of a target mRNA.

Strand: As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

Subject: As used herein, the term “subject” means any mammal, including mice, rabbits, and humans. In one embodiment, the subject is a human or non-human primate. In some embodiments, the terms “individual” or “patient” refers to a human subject.

Synthetic: As used herein, the term “synthetic” refers to a nucleic acid or other molecule that is artificially synthesized (e.g., using a machine (e.g., a solid-state nucleic acid synthesizer)) or that is otherwise not derived from a natural source (e.g., a cell or organism) that normally produces the molecule.

Targeting ligand: As used herein, the term “targeting ligand” refers to a molecule (e.g., a carbohydrate, amino sugar, cholesterol, polypeptide, or lipid) that selectively binds to a cognate molecule (e.g., a receptor) of a tissue or cell of interest and that is conjugatable to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

Tetraloop: As used herein, the term “tetraloop” refers to a loop that increases stability of an adjacent duplex formed by hybridization of flanking sequences of nucleotides. The increase in stability is detectable as an increase in melting temperature (Tₘ) of an adjacent stem duplex that is higher than the Tₘ of the adjacent stem duplex expected, on average, from a set of loops of comparable length consisting of randomly selected sequences of nucleotides. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. In some embodiments, a tetraloop may stabilize a base pair in an adjacent stem duplex by stacking interactions. In addition, interactions among the nucleotides in a tetraloop include but are not limited to non-Watson-Crick base-pairing, stacking interactions, hydrogen bonding, and contact interactions (Cheong et al., NATURE, 1990 346(6285):680-82; Heus and Pardi, SCIENCE, 1991, 253(5016):191-94). In some embodiments, a tetraloop comprises or consists of 3 to 6 nucleotides and is typically 4 to 5 nucleotides. In certain embodiments, a tetraloop comprises or consists of three, four, five, or six nucleotides, which may or may not be modified (e.g., which may or may not be conjugated to a targeting moiety). In one embodiment, a tetraloop consists of four nucleotides. Any nucleotide may be used in the tetraloop and standard IUPAC-IUB symbols for such nucleotides may be used as described in Cornish-Bowden, NUCL. ACIDS RES., 1985, 13:3021-3030. For example, the letter “N” may be used to mean that any base may be in that position, the letter “R” may be used to show that A (adenine) or G (guanine) may be in that position, and “B” may be used to show that C (cytosine), G (guanine), or T (thymine) may be in that position. Examples of tetraloops include the UNCG family of tetraloops (e.g., UUCG), the GNRA family of tetraloops (e.g., GAAA), and the CUUG tetraloop (Woese et al., PROC NATL ACAD SCI USA., 1990, 87(21):8467-71; Antao et al., NUCLEIC ACIDS RES., 1991, 19(21):5901-5). Examples of DNA tetraloops include the d(GNNA) family of tetraloops (e.g., d(GTTA)), the d(GNRA) family of tetraloops, the d(GNAB) family of tetraloops, the d(CNNG) family of tetraloops, and the d(TNCG) family of tetraloops (e.g., d(TTCG)). See, e.g., Nakano et al. BIOCHEMISTRY, 2002, 41(48):14281-14292; Shinji et al., NIPPON KAGAKKAI KOEN YOKOSHU, Vol 78^{th}, No. 2, page 731 (2000), which are incorporated by reference herein for their relevant disclosures. In some embodiments, the tetraloop is contained within a nicked tetraloop structure.

Treat: As used herein, the term “treat” refers to the act of providing care to a subject in need thereof, e.g., through the administration a therapeutic agent (e.g., an oligonucleotide) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

### II. Oligonucleotide-Based Inhibitors

i. ALDH2 Targeting Oligonucleotides

In some embodiments, an oligonucleotide described herein has a guide (antisense) strand having a sequence UAAACUGAGUUUCAUCCACCGG (SEQ ID NO: 1). In some embodiments, a sense strand is provided that forms a duplex with the antisense strand. In some embodiments, the sense strand comprises a stem-loop at its 3′-end. In certain embodiments, the sense strand comprises (e.g., at its 3′-end) a stem-loop set forth as: S1-L-S2, in which S1 is complementary to S2, and in which L forms a loop between S1 and S2 in a range of 2 to 6 nucleotides in length. In some embodiments, a duplex formed between S1 and S2 is 4, 5, 6, 7, or 8 base pairs in length. In some embodiments, a loop (L) of a stem-loop is a tetraloop (e.g., within a nicked tetraloop structure). A tetraloop may contain ribonucleotides, modified nucleotides, and/or combinations thereof. Typically, a tetraloop has 4 to 5 nucleotides. However, in some embodiments, a tetraloop comprises or consists of 3 to 6 nucleotides, and typically consists of 4 nucleotides. In certain embodiments, a tetraloop comprises or consists of three, four, five, or six nucleotides.

In some embodiments, the oligonucleotide described herein has a sense strand of sequence GGUGGAUGAAACUCAGUUUAGCAGCCGAAAGGCUGC (SEQ ID NO: 2), or a pharmaceutically acceptable salt thereof. In some embodiments, the oligonucleotide comprises an antisense strand of sequence UAAACUGAGUUUCAUCCACCGG (SEQ ID NO: 1) and a sense strand of sequence GGUGGAUGAAACUCAGUUUAGCAGCCGAAAGGCUGC (SEQ ID NO: 2), or a pharmaceutically acceptable salt thereof

ii. Oligonucleotide Modifications

In some embodiments, oligonucleotides of the present disclosure may include one or more suitable modifications. In some embodiments, a modified nucleotide has a modification in its base (or nucleobase), the sugar (e.g., ribose, deoxyribose), or the phosphate group. In some embodiments of the oligonucleotides provided herein, all, or substantially all of the nucleotides of an oligonucleotide are modified. In certain embodiments, more than half of the nucleotides are modified. In certain embodiments, less than half of the nucleotides are modified.

Chemical modification of such RNAi oligonucleotides is essential to fully harness the therapeutic potential of this class of molecules. Various chemical modifications have been developed and applied to RNAi oligonucleotides to improve their pharmacokinetics and pharmacodynamics properties (Deleavey and Damha, CHEM BIOL., 2012, 19:937-954), and to block innate immune activation (Judge et al., MOL THER., 2006, 13:494-505). One of the most common chemical modifications is to the 2′-OH of the furanose sugar of the ribonucleotides because of its involvement in the nuclease degradation. Fully chemically modified siRNAs with a combination of 2′-O-methyl (2′-OMe) and 2′-deoxy-2′-fluoro (2′-F) throughout the entire duplex have been reported and have demonstrated excellent stability and RNAi activity (Morrissey et al., HEPATOLOGY, 2005, 41:1349-1356; Allerson et al., J MED CHEM., 2005, 48:901-904; Hassler et al., NUCLEIC ACID RES., 2018, 46:2185-2196). More recently, N-acetylgalactosamine (GalNAc) conjugated chemically modified siRNAs have shown effective asialoglycoprotein receptor (ASGPr)-mediated delivery to liver hepatocytes in vivo (Nair et al., J AM CHEM SOC., 2014, 136:16958-961). Several GalNAc conjugated RNAi platforms including the GalNAc dicer-substrate conjugate (GalXC) platform, have advanced into clinical development for treating a wide range of human diseases.

One major concern with using chemically modified nucleoside analogues in the development of oligonucleotide-based therapeutics, including RNAi GalNAc conjugates, is the potential toxicity associated with the modifications. The therapeutic oligonucleotides could slowly degrade in patients, releasing nucleoside analogues that could be potentially phosphorylated and incorporated into cellular DNA or RNA. In the field of antivirus therapeutics, toxicity has emerged during the clinical development of many small molecule nucleotide inhibitors (Feng et al., ANTIMICROBIAL AGENTS AND CHEMOTHERAPY, 2016, 60:806-817). 2′-F modification of fully phosphorothioated antisense oligonucleotide has been reported to cause cellular protein reduction and double-stranded DNA breaks resulting in acute hepatotoxicity in vivo (Shen et al., NUCLEIC ACID RES., 2015, 43:4569-4578; Shen et al., NUCLEIC ACID RES., 2018, 46:2204-2217). No evidence has been observed so far for such liability of 2′-F modification in the context of RNAi oligonucleotides (Janas et al., NUCLEIC ACID THER., 2016, 26:363-371; Janas et al., NUCLEIC ACID THER., 2016, 27:11-22). Moreover, 2′-F siRNA have been well tolerated in clinical trials. Nonetheless, it is still desirable to minimize the use of unnatural nucleoside analogues such as 2′-F modified nucleosides in therapeutic RNA oligonucleotides.

Unlike 2′-deoxy-2′-fluoro RNA, 2′-O-Methyl RNA is a naturally occurring modification of RNA found in tRNA and other small RNAs that arise as a post-transcriptional modification. It is also known that the bulkier 2′-O-Methyl modification confers better metabolic stability as compared to the less bulky 2′-F modification. Therefore, 2′-OMe is preferable to 2′-F in terms of stability and tolerability. However, the bulkier 2′-OMe has been shown to interfere with RNA protein binding and inhibit RNAi activity if not positioned properly in the sequence of siRNA (Chiu et al., RNA, 2003, 9:1034-1048; Prakash et al., J. MED CHEM., 2005, 48:4247-4253; Zheng et al., FASEB J., 2013, 27:4017-4026).

To further reduce the 2′-F content and increase the 2′-OMe content concomitantly so that the stability and tolerability can be improved without compromising RNAi activity, fine-tuning of the positions of the 2′-OMe and 2′-F (modification patterns) is necessary in DsiRNA conjugates that have already shown good potency and duration. A recent report has attempted to optimize modification patterns of a 21/23mer siRNA GalNAc conjugate platform (Foster et al., MOL THER., 2018, 26:708-17). That report, however, did not identify patterns of 2′-OMe and 2′-F that confer an oligonucleotide with high potency and duration as disclosed herein, including positions having poor tolerability to 2′-OMe substitution. Nor did that report identify advanced designs with minimal 2′-F content specifically for triloop, pentaloop and tetraloop GalXC platforms as disclosed herein.

It has been surprisingly found that when the sequence contains more fluorine (e.g., fU), defluorination impurities showed up in the product on HPLC, and modification patterns with reduced fluorine content (mainly with no fU) showed no defluorination side product after cleavage and deprotection, thus, unexpectedly enhanced manufacturing yield and reduced impurity.

In certain embodiments, oligonucleotide of the present disclosure is a double stranded oligonucleotide comprising a sense strand of SEQ ID NO: 3 (DP1663P), and an antisense strand selected from SEQ ID NO: 4 (DP17232G), 5 (DP16279G), 6 (DP16281G) and 7 (DP13488G), or a pharmaceutically acceptable salt thereof, of Table 1. In certain embodiments, oligonucleotide of the present disclosure is a double stranded oligonucleotide comprising a sense strand of SEQ TD NO: 8 (DP11518P), and an antisense strand of SEQ TD NO: 9 (DP11674G), or a pharmaceutically acceptable salt thereof. In certain embodiments, the oligonucleotide comprises a sense strand of SEQ TD NO: 3, and an antisense strand of SEQ ID NO: 4. In certain embodiments, the oligonucleotide comprises a sense strand of SEQ TD NO: 3, and an antisense strand of SEQ TD NO: 5. In certain embodiments, the oligonucleotide comprises a sense strand of SEQ TD NO: 3, and an antisense strand of SEQ TD NO: 6. In certain embodiments, the oligonucleotide comprises a sense strand of SEQ ID NO: 3, and an antisense strand of SEQ TD NO: 7.

TABLE 1

| SEQ | | |
|---|---|---|
| ID | Ref. | |
| No: | No | Sequence 5′-3′ |
| 3 | DP11663P | [mGs][mG][mU][mG][mG][mA][mU] |
| | | [fG][fA][fA][[A][mC][mU][mC] |
| | | [mA][mG][mU][mU][mU][mA][mG] |
| | | [mC][mA][mG][mC][mC][mG] |
| | | [ademA-GalNAc][ademA-GalNAc] |
| | | [ademA-GalNAc][mG][mG][mC] |
| | | [mU][mG][mC] |
| 4 | DP17232G | [MePhosphonate-4O-mUs] |
| | | [fAs][fAs][fA][mC][mU][fG][mA] |
| | | [mG][mU][mU][mU][mC][fA][mU] |
| | | [mC][mC][mA][mC][mCs][mGs][mG] |
| 5 | DP16279G | [MePhosphonate-4O-mUs] |
| | | [fAs][fAs][fA][mC][mU][fG][mA] |
| | | [mG][fU][mU][mU][mC][fA][mU] |
| | | [mC][mC][mA][mC][mCs][mGs][mG] |
| 6 | DP16281G | [MePhosphonate-4O-mUs] |
| | | [fAs][fAs][fA][fC][mU][fG][mA] |
| | | [mG][mU][mU][mU][mC][fA][mU] |
| | | [mC][mC][mA][mC][mCs][mGs][mG] |
| 7 | DP13488G | [MePhosphonate-4O-mUs] |
| | | [fAs][fAs][fA][fC][mU][fG][mA] |
| | | [mG][fU][mU][mU][mC][fA][mU] |
| | | [mC][mC][mA][mC][mCs][mGs][mG] |
| 8 | DP11518P | [mGs][mG][fU][mG][fG][mA][mU] |
| | | [fG][mA][fA][mA][fC][fU][mC] |
| | | [fA][mG][fU][mU][mU][mA][mG] |
| | | [mC][mA][mG][mC][mC][mG] |
| | | [ademA-GalNAc][ademA-GalNAc] |
| | | [ademA-GalNAc][mG][mG][mC][mU] |
| | | [mG][mC] |
| 9 | DP11674G | [MePhosphonate-4O-mUs] |
| | | [fAs][fA][fA][fC][mU][fG][mA] |
| | | [mG][fU][mU][mU][mC][fA][mU] |
| | | [fC][mC][mA][fC][mCs][mGs][mG] |

In certain aspects, oligonucleotide of a present disclosure is a double stranded oligonucleotide comprising a sense strand:
5′ mG-S-mG-mU-mG-mG-mA-mU-fG-fA-fA-fA-mC-mU-mC-mA-mG-mU-mU-mU-mA-mG-mC-mA-mG-mC-mC-mG-[ademA-GalNAc]-[ademA-GalNAc]-[ademA-GalNAc]-mG-mG-mC-mU-mG-mC 3′ (SEQ ID NO: 3), and
an antisense strand:
5′ [MePhosphonate-4O-mU]-S-fA-S-fA-S-fA-fC-mU-fG-mA-mG-mU-mU-mU-mC-fA-mU-mC-mC-mA-mC-mC-S-mG-S-mG 3′ (SEQ ID NO: 6); or a pharmaceutically acceptable salt thereof
   wherein:
“-” between nucleosides represent a phosphodiester internucleoside linkage;
“—S—” between nucleosides represent a phosphorothioate internucleoside linkage;
mA represents 2′-O-methyladenosine ribonucleoside;
mG represents 2′-O-methylguanosine ribonucleoside;
mC represents 2′-O-methylcytidine ribonucleoside;
mU represents 2′-O-methyluridine ribonucleoside;
fA represents 2′-fluoro-adenosine deoxyribonucleoside;
fG represents 2′-fluoro-guanosine deoxyribonucleoside;
fC represents 2′-fluoro-cytidine deoxyribonucleoside;
fU represents 2′-fluoro-uridine deoxyribonucleoside;
[ademA-GalNAc] represents:

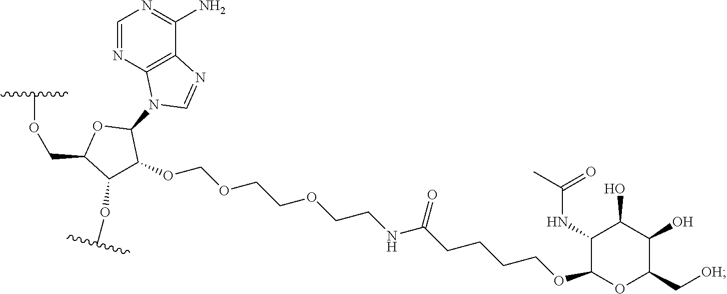 [SMILES: *OC[C@H]1O[C@@H](n2cnc3c(N)ncnc32)[C@H](OCOCCOCCNC(=O)CCCCO[C@@H]2O[C@H](CO)[C@H](O)[C@H](O)[C@H]2NC(C)=O)[C@@H]1O*]

[MePhosphonate-4O-mU] represents:

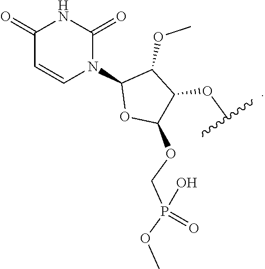 [SMILES: *O[C@@H]1[C@@H](OCP(=O)(O)OC)O[C@@H](n2ccc(=O)[nH]c2=O)[C@@H]1OC]

In certain embodiments, the double stranded oligonucleotide is a sodium salt.

a. Sugar Modifications

In some embodiments, a modified sugar (also referred to herein as a sugar analog) includes a modified deoxyribose or ribose moiety. In some embodiments, a nucleotide modification in a sugar comprises a 2′-modification. A 2′-modification may be 2′-aminoethyl, 2′-fluoro, 2′-O-methyl, 2′-O-methoxyethyl, or 2′-deoxy-2′-fluoro-β-d-arabinonucleic acid. Typically, the modification is 2′-fluoro or 2′-O-methyl. In some embodiments, a modification in a sugar comprises a modification of the sugar ring, which may comprise modification of one or more carbons of the sugar ring.

In some embodiments, one or more of the following positions are modified with a 2′-O-methyl: positions 1-7 and 12-36 of the sense strand and/or positions 1, 6, 8-13 and 15-22 of the antisense strand. In some embodiments, all of positions 1-7 and 12-36 of the sense strand and positions 1, 6, 8-13 and 15-22 of the antisense strand are modified with a 2′-O-methyl. In some embodiments, one or more of the following positions are modified with a 2′-fluoro: positions 8-11 of the sense strand and/or positions 2-5, 7 and 14 of the antisense strand. In some embodiments, all of positions 8-11 of the sense strand and positions 2-5, 7 and 14 of the antisense strand are modified with a 2′-fluoro.

In some embodiments, the terminal 3′-end group (e.g., a 3′-hydroxyl) is a phosphate group or other group, which can be used, for example, to attach linkers, adapters, or labels.

b. 5′ Terminal Phosphates

In some embodiments, 5′-terminal phosphate groups of oligonucleotides enhance the interaction with Argonaut 2. However, oligonucleotides comprising a 5′-phosphate group may be susceptible to degradation via phosphatases or other enzymes, which can limit their bioavailability in vivo. In some embodiments, oligonucleotides include analogs of 5′ phosphates that are resistant to such degradation.

In some embodiments, an oligonucleotide has a phosphate analog at a 4′-carbon position of the sugar (referred to as a “4′-phosphate analog”). See, for example, International Application No. PCT/US2017/049909, entitled 4′-Phosphate Analogs and Oligonucleotides Comprising the Same, filed on Sep. 1, 2017, the contents of which relating to phosphate analogs are incorporated herein by reference.

In some embodiments, an oligonucleotide provided herein comprises a 4′-phosphate analog at a 5′-terminal nucleotide. In some embodiments, a phosphate analog is an oxymethylphosphonate, in which the oxygen atom of the oxymethyl group is bound to the sugar moiety (e.g., at its 4′-carbon) or analog thereof. In other embodiments, a 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, a 4′-phosphate analog is an oxymethylphosphonate.

In certain embodiments, a phosphate analog attached to the oligonucleotide is a methoxy phosphonate (MOP). In certain embodiments, a phosphate analog attached to the oligonucleotide is a 5′ monomethyl protected MOP. In some embodiments, the following uridine nucleotide comprising a phosphate analog may be used, e.g., at the first position of a guide (antisense) strand:

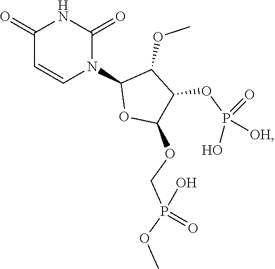 [SMILES: CO[C@@H]1[C@H](OP(=O)(O)O)[C@@H](OCP(=O)(O)OC)O[C@H]1n1ccc(=O)[nH]c1=O]

which modified nucleotide is referred to as [MePhosphonate-4O-mU] or 5′-Methoxy, Phosphonate-4′-oxy-2′-O-methyluridine.

c. Modified Internucleotide Linkages

In some embodiments, phosphate modifications or substitutions may result in an oligonucleotide that comprises at least one (e.g., at least 1, at least 2, at least 3 or at least 5) modified internucleotide linkage. In some embodiments, any one of the oligonucleotides disclosed herein comprises 1, 2, 3, 4, 5, 6, 7, 8, 9, or 10 modified internucleotide linkages. In some embodiments, at least one modified internucleotide linkage of any one of the oligonucleotides as disclosed herein is a phosphorothioate linkage.

In some embodiments, the oligonucleotide comprises at least one modified internucleotide linkage. In some embodiments, the at least one modified internucleotide linkage is a phosphorothioate linkage. In some embodiments, the oligonucleotide has a phosphorothioate linkage between one or more of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand. In some embodiments, the oligonucleotide has a phosphorothioate linkage between each of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

iii. Targeting Ligands

In some embodiments, oligonucleotides disclosed herein are modified to facilitate targeting of a particular tissue, cell, or organ, e.g., to facilitate delivery of the oligonucleotide to the liver. In certain embodiments, oligonucleotides disclosed herein may be modified to facilitate delivery of the oligonucleotide to the hepatocytes of the liver.

In some embodiments, an oligonucleotide comprises a nucleotide that is conjugated to one or more targeting ligands. In certain embodiments, the targeting ligand is one or more GalNAc moieties. GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). In some embodiments, conjugation of GalNAc moieties to oligonucleotides of the instant disclosure is used to target these oligonucleotides to the ASGPR expressed on these hepatocyte cells. In some embodiments, an oligonucleotide of the instant disclosure is conjugated directly or indirectly to a monovalent GalNAc moiety. In some embodiments, an oligonucleotide of the instant disclosure is conjugated to one or more bivalent GalNAc, trivalent GalNAc, or tetravalent GalNAc moieties. In some embodiments, an oligonucleotide of the instant disclosure is conjugated to trivalent GalNAc moieties.

In some embodiments, an oligonucleotide herein comprises a monovalent GalNAc attached to an adenine nucleotide, referred to as [ademA-GalNAc] or 2′-aminodiethoxymethanol-Adenine-GalNAc, as depicted below.

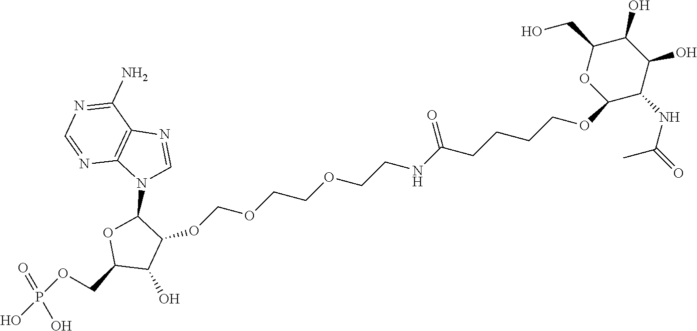 [SMILES: CC(=O)N[C@@H]1[C@@H](OCCCCC(=O)NCCOCCOCO[C@@H]2[C@H](O)[C@@H](COP(=O)(O)O)O[C@H]2n2cnc3c(N)ncnc32)O[C@@H](CO)[C@@H](O)[C@H]1O]

In some embodiments, all three adenosine nucleotides of the -GAAA- of the oligonucleotide are each conjugated to a GalNAc moiety. In some embodiments, 3 nucleotides of the loop (L) of the stem-loop are each conjugated to a separate GalNAc.

Appropriate methods or chemistry (e.g., click chemistry) may be used to link a targeting ligand to a nucleotide. In some embodiments, the linker is a labile linker. However, in other embodiments, the linker is more stable. In some embodiments, a targeting ligand is conjugated to a nucleotide using a click linker. In some embodiments, an acetal-based linker is used to conjugate a targeting ligand to a nucleotide of an oligonucleotide described herein. Acetal-based linkers are disclosed, for example, in International Patent Application Publication Number WO2016100401 A1, which published on Jun. 23, 2016, and the contents of which relating to such linkers are incorporated herein by reference.

In some embodiments, it is desirable to target an oligonucleotide that reduces the expression of ALDH2 to the hepatocytes of the liver of a subject. Any suitable hepatocyte targeting moiety may be used for this purpose.

GalNAc is a high affinity ligand for asialoglycoprotein receptor (ASGPR), which is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins). Conjugation (either indirect or direct) of GalNAc moieties to oligonucleotides of the instant disclosure may be used to target these oligonucleotides to the ASGPR expressed on these hepatocyte cells.

In some embodiments, an oligonucleotide of the instant disclosure is conjugated directly or indirectly to a monovalent GalNAc. In some embodiments, the oligonucleotide is conjugated directly or indirectly to more than one monovalent GalNAc (i.e., is conjugated to 2, 3, or 4 monovalent GalNAc moieties, and is typically conjugated to 3 or 4 monovalent GalNAc moieties). In some embodiments, an oligonucleotide of the instant disclosure is conjugated to one or more bivalent GalNAc, trivalent GalNAc, or tetravalent GalNAc moieties.

### III. Pharmaceutically Acceptable Salts

In some embodiments, an oligonucleotide of the present disclosure is in a form of a pharmaceutically acceptable salt. Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. PHARMA SCI., 1977, 66:1-19). The base addition salt forms can be prepared by contacting the free acid form with enough of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates, and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts forms may also be prepared with a pharmaceutically acceptable cation. Suitable pharmaceutically acceptable cations are well known to those skilled in the art and include alkaline, alkaline earth, ammonium, and quaternary ammonium cations. Carbonates or hydrogen carbonates are also possible.

For oligonucleotides, preferred pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

In one preferred embodiment, the oligonucleotides of disclosure is in the form of a sodium salt of the oligonucleotide. In some embodiments, oligonucleotide of the present disclosure is a double stranded oligonucleotide, where the double stranded oligonucleotide is in the form of a sodium salt.

### IV. Formulations

Various formulations have been developed to facilitate oligonucleotide use. For example, oligonucleotides can be delivered to a subject or a cellular environment using a formulation that minimizes degradation, facilitates delivery and/or uptake, or provides another beneficial property to the oligonucleotides in the formulation. In some embodiments, provided herein are compositions comprising oligonucleotides (e.g., single-stranded, or double-stranded oligonucleotides) to reduce the expression of ALDH2. Such compositions can be suitably formulated such that when administered to a subject, either into the immediate environment of a target cell or systemically, a sufficient portion of the oligonucleotides enter the cell to reduce ALDH2 expression. Any of a variety of suitable oligonucleotide formulations can be used to deliver oligonucleotides for the reduction of ALDH2 as disclosed herein. In some embodiments, an oligonucleotide is formulated in buffer solutions such as phosphate-buffered saline solutions, liposomes, micellar structures, and capsids. In some embodiments, naked oligonucleotides or conjugates thereof are formulated in water or in an aqueous solution (e.g., water with pH adjustments). In some embodiments, naked oligonucleotides or conjugates thereof are formulated in basic buffered aqueous solutions (e.g., PBS).

In certain aspects, the present disclosure presents a composition comprising a double stranded oligonucleotide, wherein the double stranded oligonucleotide comprises a sense strand:
5′mG-S-mG-mU-mG-mG-mA-mU-fG-fA-fA-fA-mC-mU-mC-mA-mG-mU-mU-mU-mA-mG-mC-mA-mG-mC-mC-mG-[ademA-GalNAc]-[ademA-GalNAc]-[ademA-GalNAc]-mG-mG-mC-mU-mG-mC 3′ (SEQ ID NO: 3), and
an antisense strand:
5′ [MePhosphonate-4O-mU]-S-fA-S-fA-S-fA-fC-mU-fG-mA-mG-mU-mU-mU-mC-fA-mU-mC-mC-mA-mC-mC-S-mG-S-mG 3′ (SEQ ID NO: 6); or a pharmaceutically acceptable salt thereof,
   wherein:
“-” between nucleosides represent a phosphodiester internucleoside linkage;
“—S—” between nucleosides represent a phosphorothioate internucleoside linkage;
mA represents 2′-O-methyladenosine ribonucleoside;
mG represents 2′-O-methylguanosine ribonucleoside;
mC represents 2′-O-methylcytidine ribonucleoside;
mU represents 2′-O-methyluridine ribonucleoside;
fA represents 2′-fluoro-adenosine deoxyribonucleoside;
fG represents 2′-fluoro-guanosine deoxyribonucleoside;
fC represents 2′-fluoro-cytidine deoxyribonucleoside;
fU represents 2′-fluoro-uridine deoxyribonucleoside;
[ademA-GalNAc] represents

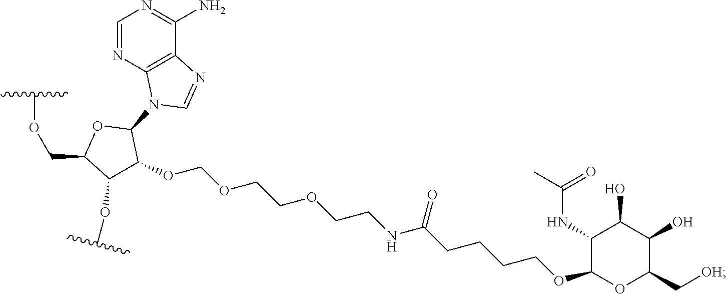 [SMILES: *OC[C@H]1O[C@@H](n2cnc3c(N)ncnc32)[C@H](OCOCCOCCNC(=O)CCCCO[C@@H]2O[C@H](CO)[C@H](O)[C@H](O)[C@H]2NC(C)=O)[C@@H]1O*]

[MePhosphonate-4O-mU] represents

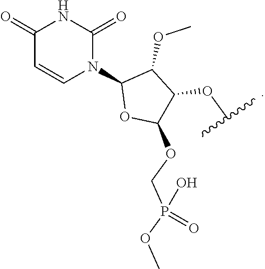 [SMILES: *O[C@@H]1[C@@H](OCP(=O)(O)OC)O[C@@H](n2ccc(=O)[nH]c2=O)[C@@H]1OC]

In some embodiments, a pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Typically, the route of administration is intravenous or subcutaneous.

Pharmaceutical compositions suitable for injectable use include sterile aqueous solutions (where water soluble) or dispersions and sterile powders for the extemporaneous preparation of sterile injectable solutions or dispersions. For intravenous or subcutaneous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. In many cases, it will be preferable to include isotonic agents, for example, sugars, polyalcohols such as mannitol, sorbitol, and sodium chloride in the composition. Sterile injectable solutions can be prepared by incorporating the oligonucleotides in a required amount in a selected solvent with one or a combination of ingredients enumerated above, as required, followed by filtered sterilization.

In some embodiments, a composition may contain at least about 0.1% of the therapeutic agent (e.g., an oligonucleotide for reducing ALDH2 expression) or more, although the percentage of the active ingredient(s) may be between about 1% and about 80% or more of the weight or volume of the total composition. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

Even though several embodiments are directed to liver-targeted delivery of any of the oligonucleotides disclosed herein, targeting of other tissues is also contemplated.

### IV. Methods of Use

i. Reducing ALDH2 Expression in Cells

In some embodiments, methods are provided for delivering to a cell an effective amount any one of oligonucleotides disclosed herein for purposes of reducing expression of ALDH2 in the cell. Methods provided herein are useful in any appropriate cell type. In some embodiments, a cell is any cell that expresses ALDH2 (e.g., hepatocytes, macrophages, monocyte-derived cells, prostate cancer cells, cells of the brain, endocrine tissue, bone marrow, lymph nodes, lung, gall bladder, liver, duodenum, small intestine, pancreas, kidney, gastrointestinal tract, bladder, adipose and soft tissue, and skin). In some embodiments, the cell is a primary cell that has been obtained from a subject and that may have undergone a limited number of a passages, such that the cell substantially maintains its natural phenotypic properties. In some embodiments, a cell to which the oligonucleotide is delivered is ex vivo or in vitro (i.e., can be delivered to a cell in culture or to an organism in which the cell resides). In specific embodiments, methods are provided for delivering to a cell an effective amount any one of the oligonucleotides disclosed herein for purposes of reducing expression of ALDH2 solely in hepatocytes.

The consequences of inhibition can be confirmed by an appropriate assay to evaluate one or more properties of a cell or subject, or by biochemical techniques that evaluate molecules indicative of ALDH2 expression (e.g., RNA, protein). In some embodiments, the extent to which an oligonucleotide provided herein reduces levels of expression of ALDH2 is evaluated by comparing expression levels (e.g., mRNA or protein levels of ALDH2 to an appropriate control (e.g., a level of ALDH2 expression in a cell or population of cells to which an oligonucleotide has not been delivered or to which a negative control has been delivered).

In some embodiments, an appropriate control level of ALDH2 expression may be a predetermined level or value, such that a control level need not be measured every time. The predetermined level or value can take a variety of forms. In some embodiments, a predetermined level or value can be single cut-off value, such as a median or mean.

In some embodiments, administration of an oligonucleotide as described herein results in a reduction in the level of ALDH2 expression in a cell. In some embodiments, the reduction in levels of ALDH2 expression may be a reduction to 1% or lower, 5% or lower, 10% or lower, 15% or lower, 20% or lower, 25% or lower, 30% or lower, 35% or lower, 40% or lower, 45% or lower, 50% or lower, 55% or lower, 60% or lower, 70% or lower, 80% or lower, or 90% or lower compared with an appropriate control level of ALDH2. The appropriate control level may be a level of ALDH2 expression in a cell or population of cells that has not been contacted with an oligonucleotide as described herein. In some embodiments, the effect of delivery of an oligonucleotide to a cell according to a method disclosed herein is assessed after a finite period. For example, levels of ALDH2 may be analyzed in a cell at least 8 hours, 12 hours, 18 hours, 24 hours; or at least one, two, three, four, five, six, seven, or fourteen days after introduction of the oligonucleotide into the cell.

ii. Treatment Methods

Aspects of the disclosure relate to methods for reducing ADH1B, ADH1C and ALDH2 expression for the treatment of alcoholism in a subject. In certain embodiments, the disclosure relates to methods for reducing ALDH2 expression for the treatment of alcoholism in a subject. In some embodiments, the methods may comprise administering to a subject in need thereof an effective amount of any one of the oligonucleotides disclosed herein. Such treatments could be used, for example, to decrease ethanol tolerance in a subject, thereby inhibiting ethanol intake by the subject (e.g., by decreasing the desire of the subject to consume ethanol). The present disclosure provides for both prophylactic and therapeutic methods of treating a subject at risk of (or susceptible to) alcoholism and/or a disease or disorder associated with alcoholism.

In certain aspects, the disclosure provides a method for preventing in a subject, a disease or disorder as described herein by administering to the subject a therapeutic agent (e.g., an oligonucleotide or vector or transgene encoding same). In some embodiments, the subject to be treated is a subject who will benefit therapeutically from a reduction in the amount of ALDH2 protein, e.g., in the liver.

Methods described herein typically involve administering to a subject an effective amount of an oligonucleotide, that is, an amount capable of producing a desirable therapeutic result. A therapeutically acceptable amount may be an amount that can treat a disease or disorder. The appropriate dosage for any one subject will depend on certain factors, including the subject's size, body surface area, age, the composition to be administered, the active ingredient(s) in the composition, time and route of administration, general health, and other drugs being administered concurrently.

In some embodiments, a subject is administered any one of the compositions disclosed herein either enterally (e.g., orally, by gastric feeding tube, by duodenal feeding tube, via gastrostomy or rectally), parenterally (e.g., subcutaneous injection, intravenous injection or infusion, intra-arterial injection or infusion, intramuscular injection), topically (e.g., epicutaneous, inhalational, via eye drops, or through a mucous membrane), or by direct injection into a target organ (e.g., the liver of a subject). Typically, oligonucleotides disclosed herein are administered intravenously or subcutaneously.

In some embodiments, oligonucleotides are administered at a dose in a range of 0.1 mg/kg to 25 mg/kg (e.g., 1 mg/kg to 5 mg/kg). In some embodiments, oligonucleotides are administered at a dose in a range of 0.1 mg/kg to 5 mg/kg or in a range of 0.5 mg/kg to 5 mg/kg.

In some embodiments the oligonucleotides herein are administered alone or in combination. In some embodiments the oligonucleotides herein are administered in combination concurrently, sequentially (in any order), or intermittently. For example, two oligonucleotides may be co-administered concurrently. Alternatively, one oligonucleotide may be administered and followed any amount of time later (e.g., one hour, one day, one week or one month) by the administration of a second oligonucleotide. In certain embodiments, the oligonucleotides herein can be administered in combination with Disulfiram.

As a non-limiting set of examples, the oligonucleotides of the instant disclosure would typically be administered once per year, twice per year, quarterly (once every three months), bi-monthly (once every two months), monthly, or weekly.

In some embodiments, the subject to be treated is a human or non-human primate or other mammalian subject. Other exemplary subjects include domesticated animals such as dogs and cats; livestock such as horses, cattle, pigs, sheep, goats, and chickens; and animals such as mice, rats, guinea pigs, and hamsters.

### EXAMPLES

### Example 1: Impact of Substitution on In Vivo Potency

### In Vivo Murine Experimentation

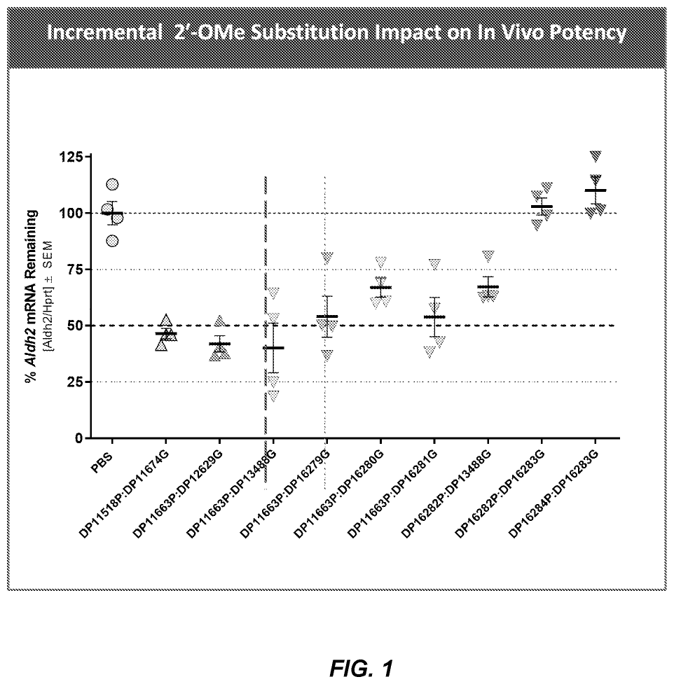

Modification patterns that would improve delivery properties while maintaining activity for reduction of ALDH2 expression in the mouse hepatocytes were analyzed. Oligonucleotides with various 2′-OMe modification patterns were analyzed for their potency by administering them subcutaneously to CD-1 mice at 3 mg/kg. Mice were euthanized on day 4 following administration. Liver samples were obtained, and RNA was extracted to evaluate ALDH2 mRNA levels by RT-qPCR. The percent ALDH2 mRNA as compared to PBS control mRNA was determined based on these measurements and is shown in FIG. 1.

### Example 2: Duration Study of GalNAc-Conjugated ALDH2 Oligonucleotides in Non-Human Primates (NHP)

This study was designed to evaluate pharmacodynamics of a single dose of GalNAc-conjugated ALDH2 oligonucleotides with different modification patterns (e.g., modification patterns that have different numbers of 2′-fluoro modifications and/or different numbers of phosphorothioate linkages in the anti-sense strand). The GalNAc-conjugated ALDH2 oligonucleotides tested in this study were: S585-AS595-M14, S585-AS595-M15, S585-AS595-M16, S585-AS595-M17, S587-AS597-M23, and S587-AS597-M24. These oligonucleotides are disclosed in international patent publication WO20119/143621, incorporated herein by reference. A single dose of the GalNAc-conjugated ALDH2 oligonucleotides were subcutaneously administered to non-human primates (n=4 for each group) at 3 mg/kg. Animals fasted overnight and serum samples and liver biopsies were collected prior to feeding the next morning. One pre-dose biopsy was collected for each animal during acclimation and three biopsies were collected 4, 8, or 12-, or 16-weeks post administration. The biopsies were divided into two sections, one was flash-frozen and stored at −80° C. and the other was processed in RNA later (Thermo Fisher Scientific) and stored at 4° C. for mRNA level analyses.

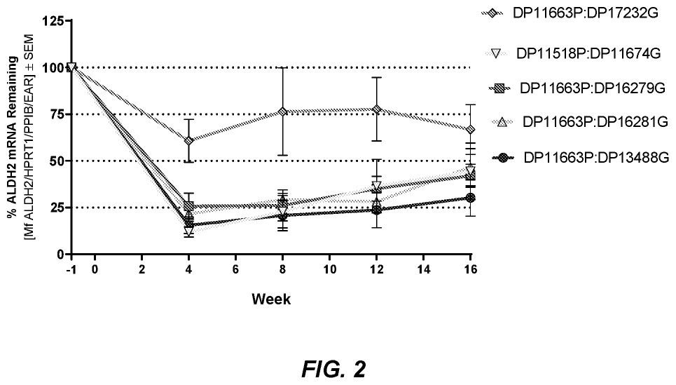

The amount of ALDH2 mRNA remaining 4-, 8-, 12-, or 16-weeks following administration, relative to the amount of ALDH2 mRNA prior to administration were analyzed by quantitative PCR (qPCR) and the results showed that four out of the six GalNAc-conjugated ALDH2 oligonucleotides achieved about 50% ALDH2 mRNA suppression and the effects maintained for three months after a single 3 mg/kg dose (FIG. 2). The results support a proposed dosing frequency of once-per-quarter or less in humans.

The serum samples were for stored liver function panel test, including Alanine Aminotransferase (ALT), Alkaline Phosphatase (ALP) Lactate Dehydrogenase (LDH), Gamma Glutamyl Transferase (GGT).

### Materials

Liver samples were homogenized in 0.75 mL phenol/guanidine based QIAzol Lysis Reagent (Qiagen, Valencia, CA) using a Tissuelyser II (Qiagen, Valencia, CA). The homogenate was extracted with 1-Bromo-3-chloropropane (Sigma-Aldrich, St. Louis, MO). RNA was extracted from 0.2 mL of the aqueous phase using the MagMax Technology (Thermo Fisher Scientific, Waltham, MA) according to the manufacturer's instructions. RNA was quantified using spectrometry at 260 and 280 nm. High-capacity cDNA reverse transcription kit (Thermo Fisher Scientific, Waltham, MA) was used to prepare cDNA. RT-qPCR assays from Integrated DNA Technologies (Coralville, IA) and reagents from Bio-Rad Laboratories (Hercules, CA) were used to measure ALDH2 mRNA level with normalization to endogenous housekeeping genes. The degree of ALDH2 mRNA expression was normalized to the PBS group (mouse studies) or to the pre-dose biopsy (NHP studies).

### Example 3: A 5-Week Study of DP11663P:DP16281G (DCR-A1203) by Subcutaneous Injection in Mice

### Summary

The objectives of this study were to determine the potential toxicity of repeat-dose (every 4 weeks; 2 doses) subcutaneous (SC) administration of DCR-A1203 in CD-1 mice and to evaluate the potential reversibility of any findings. In addition, the toxicokinetic (TK) characteristics of DCR-A1203 were determined.

Animals were dosed once every 4 weeks (Days 1 and 29) via SC injection, except for 3 animals/sex in Group 1 and 15 animals/sex in Groups 2-4 in the TK phase that only received a single dose (on Day 1). The study design is presented in Table 2.

TABLE 2
Experimental Design

| | | | | | Number of Animals | | | | | | | |
|---|---|---|---|---|---|---|---|---|---|---|---|---|
| | | Dose | Dose | Dose | Main | | Recovery | | | | | |
| Group | Test | Level | Volume | Conc. | Study | | Study^{c} | | TK Study | | PD Study | |
| No. | Material | (mg/kg)^{a} | (mL/kg) | (mg/mL)^{b} | M | F | M | F | M | F | M | F |
| 1 | Vehicle | 0 | 5 | 0 | 10 | 10 | 6 | 6 | 6 | 6 | 6 | 6 |
| 2 | DCR-A1203 | 30 | 5 | 6 | 10 | 10 | — | — | 33 | 33 | 6 | 6 |
| 3 | DCR-A1203 | 100 | 5 | 20 | 10 | 10 | 6 | 6 | 33 | 33 | 6 | 6 |
| 4 | DCR-A1203 | 300 | 5 | 60 | 10 | 10 | 6 | 6 | 33 | 33 | 6 | 6 |

— = not applicable; M = males; F = females; TK = toxicokinetic; PD = pharmacodynamic.
^{a}Based on the most recent body weight measurement. The first day of dosing was based on Day 1 body weight measurement and the last day of dosing was based on Day 29 body weight measurement.
^{b}Formulation concentrations were corrected for purity and moisture content (correction factor = 1.14).
^{c}the first day of the recovery period was Day 30.

All animals survived to the scheduled necropsies. There were no DCR-A1203-related clinical observations or effects on body weight, food consumption, hematology, clinical chemistry, urinalysis, or organ weights. There were no DCR-A1203-related ophthalmic or macroscopic findings.

DCR-A1203-related non-adverse microscopic findings noted at the terminal euthanasia included minimal to mild vacuolated/granular macrophages at the injection sites at all dose levels in males and females, minimal vacuolated/granular epithelial cells in the kidneys at all dose levels in males and in the 100 and 300 mg/kg group females, minimal vacuolated/granular hepatocytes in the liver in the 300 mg/kg group males and females, and minimal vacuolated/granular macrophages in the lymph nodes (axillary, mandibular, and mesenteric) in the 100 and/or 300 mg/kg group males and 300 mg/kg group females. These microscopic findings resembled common histopathologic features associated with oligonucleotide administration and were considered non-adverse given the absence of any changes suggestive of toxicity (e.g., degeneration/necrosis of epithelial cells or hepatocytes, or proinflammatory effects). At the recovery euthanasia, DCR-A1203-related microscopic changes were still noted at the injection sites (minimal to mild vacuolated/granular macrophages) in the 100 and 300 mg/kg group males and females and lymph nodes (axillary, mandibular, and mesenteric) in the 100 and/or 300 mg/kg group males and 300 mg/kg group females; however, there were no findings in the kidney and liver, indicating partial resolution of changes in these tissues.

DCR-A1203 concentrations were quantifiable in liver and kidney tissues at all dose levels on Days 1, 29 (24 hours post dose) and 58 (sample collected during terminal necropsy). Liver concentrations increased less-than-dose-proportionally with increase in dose level from 30 to 300 mg/kg on all evaluation days. Kidney concentrations increased nearly dose-proportionally on Days 1 and 29 and greater-than-dose proportionally on Day 58 with dose level increment from 30 to 300 mg/kg. DCR-A1203 concentrations were higher in liver than in kidney at all time points evaluated. Accumulation of DCR-A1203 in liver and kidney was not observed on Day 29, as indicated by ARC_{24 hr} values. Liver and kidney DCR A1203 concentrations decreased by greater than 96% on Day 58 as compared to Day 29 concentrations.

TABLE 3
Mean Tissue and Plasma Results, and Tissue to Plasma Ratios
for DCR-A1203 in Male and Female Mice (Sexes Combined)

| Dose | | Liver | Kidney | Plasma | Liver/Plasma | Kidney/Plasma | Liver/Kidney |
|---|---|---|---|---|---|---|---|
| (mg/kg) | Day | (ng/g) | (ng/g) | (ng/mL) | Ratio | Ratio | Ratio |
| 30 | 1 | 98000 | 22900 | 18.4 | 8100 | 1900 | 4.3 |
| 100 | | 169000 | 72200 | 36.3 | 4900 | 2100 | 2.4 |
| 300 | | 264000 | 186000 | 94.1 | 3000 | 2100 | 1.6 |
| 30 | 29 | 88500 | 17300 | 29.1 | 4500 | 1000 | 5.6 |
| 100 | | 215000 | 77800 | 41.4 | 5500 | 2100 | 3.3 |
| 300 | | 360000 | 224000 | 146 | 3600 | 2000 | 2.0 |
| 30 | 58 | 3400 | 165 | NA | NA | NA | 15 |
| 100 | | 5240 | 1200 | NA | NA | NA | 6.3 |
| 300 | | 14900 | 3600 | NA | NA | NA | 6.0 |

NA = Not applicable as no plasma sample was available for DCR-A1203 concentration analysis on Day 58.

The liver was the primary target organ for delivery and activity of DCR-A1203. Evidence of DCR-A1203 activity in the liver was demonstrated by a reduction of Aldh2 mRNA by >97% in the Day 31 study groups administered 30, 100, and 300 mg/kg relative to controls. There was no recovery of Aldh2 mRNA expression in the liver at any dose levels (Table 3). No DCR-A1203 activity was detected in the kidneys and activity in the esophagus and bone marrow was only detected in the Day 58 groups. Reduction in Aldh2 mRNA in esophagus and bone marrow was observed only in samples collected on Day 58. There was minimal but statistically significant reduction in Aldh2 mRNA in the esophagus (11.7-42.0%) and bone marrow (42.8-55.1%) on Day 58 with a downward trend on Day 31. The mechanisms for reduction are unclear given the lack of ASGPR expression in these tissues, the minimal nature of the reduction of Aldh2 mRNA relative to liver and late onset of these changes. There was no apparent difference in Aldh2 mRNA expression or DCR-A1203 activity between male and female mice.

In conclusion, administration of DCR-A1203 once every 4 weeks (2 total doses; Days 1 and 29) via subcutaneous injection to Crl:CD1(ICR) mice at dose levels of 30, 100, and 300 mg/kg was tolerated with no mortality or adverse findings. Non-adverse findings were limited to microscopic findings of minimal to mild vacuolated/granular macrophages at the injection site, minimal vacuolated/granular epithelial cells in the kidneys, minimal vacuolated/granular hepatocytes in the liver, and minimal vacuolated/granular macrophages in the axillary, mandibular, and mesenteric lymph nodes at the terminal euthanasia but only findings at the injection sites and lymph nodes were still present at the recovery euthanasia. Based on these results, the no-observed-adverse-effect level (NOAEL) was 300 mg/kg. This dose corresponded to mean AUCₗₐₛₜ values of 391,000 hr*ng/mL and mean Cₘₐₓ values of 134,000 ng/mL for sexes combined on Day 29.

### Example 4: A 5-Week Study of DP11663P:DP16281G (DCR-A1203) by Subcutaneous Injection in Monkey

### Summary

The objectives of this study were to determine the potential toxicity of DCR-A1203 when administered subcutaneously once every 28 days for a total of 2 doses, to cynomolgus monkeys, and to evaluate the potential reversibility of any findings over a 4-week recovery period. In addition, the toxicokinetic characteristics of DCR-A1203 were determined.

The study design was as follows:

TABLE 4
Experimental Design

| | | | Dose | Dose | No. of Animals^{c} | | | |
|---|---|---|---|---|---|---|---|---|
| Group | Test | Dose Level | Volume | Concentration | Main Study | | Recovery Study | |
| No. | Material | (mg/kg/dose) | (mL/kg)^{a} | (mg/mL)^{b} | Males | Females | Males | Females |
| 1 | Control | 0 | 1.5 | 0 | 4 | 4 | 2 | 2 |
| 2 | DCR-A1203 | 30 | 0.15 | 200 | 4 | 4 | — | — |
| 3 | DCR-A1203 | 100 | 0.5 | 200 | 4 | 4 | 2 | 2 |
| 4 | DCR-A1203 | 300 | 1.5 | 200 | 4 | 4 | 2 | 2 |

— = not applicable.
^{a}Based on the most recent body weight measurement.
^{b}The concentration was based upon full siRNA duplex content.
^{c}Main Study animals were euthanized on Day 31. Recovery animals were euthanized on Day 57.

The following parameters and end points were evaluated in this study: mortality, clinical observations, body weights, food consumption, ophthalmology, electrocardiology exams, clinical pathology parameters (hematology, coagulation, clinical chemistry, and urinalysis), bioanalysis and toxicokinetic parameters, tissue pharmacodynamic analysis (target mRNA expression), complement (C3a and Bb), cytokines and chemokines, organ weights, and macroscopic and microscopic examinations.

Repeat-dose administration of DCR-A1203 produced no changes in clinical observations, body weights, qualitative food consumption, ophthalmology parameters, electrocardiology parameters, coagulation parameters, complement factors C3a and Bb, or macroscopic necropsy observations during the study.

DCR-A1203-related changes in hematology and clinical chemistry parameters were observed at ≥100 mg/kg/dose and included minimally to moderately increased neutrophils (1.98× to 7.55× baseline [range Days 2 and 30]), mildly decreased eosinophils (0.15× to 0.28× baseline on Day 30, except for males at 100 mg/kg/dose) and increased alkaline phosphatase (1.38× to 1.75× [range Days 2 and 30]). Additionally, there was DCR-A1203-related mildly increased alanine aminotransferase in a single female animal at 300 mg/kg/dose, which correlated with microscopic findings of minimal single cell hepatocellular necrosis. By recovery, the DCR-A1203-related group changes in clinical pathology parameters approximated control values, indicating reversibility. No clinical pathology changes were considered adverse.

At 8 and/or 24 hours post dose on Days 1 and 29, both sexes at ≥30 mg/kg/dose had increased mean Interleukin (IL)-6 concentrations relative to control group means, which had fully resolved by the pre-dose time point of Day 29. These increases were generally comparable in magnitude between 30 and 100 mg/kg/dose but more pronounced at 300 mg/kg/dose. Increases in IL-6 were considered minimal to mild (ranging from 1.7× to 7.6×) at 8 hours post dose Day 1 and Day 29 and minimal to moderate (ranging from 2.0× to 47.5×) at 24 hours post dose Day 1 and 29. They were typically more pronounced in males at all dose levels at 8 hours post dose on Day 1 and Day 29, and in males at 300 mg/kg/dose (8 and 24 hours post each dose), but were more pronounced in females at 30 and 100 mg/kg/dose at 24 hours post dose Day 1 and Day 29. The increased IL-6 was indicative of a pro-inflammatory response and at ≥100 mg/kg/dose on Days 2 and 30 correlated with increased incidence in minimally increased alkaline phosphatase activity. The increases in IL-6 were not considered adverse.

At terminal euthanasia, DCR-A1203-related higher mean absolute and/or relative (to brain) liver weights were observed in males administered 300 mg/kg/dose; hepatocellular hypertrophy was observed as a microscopic correlate. At recovery euthanasia, no DCR-A1203-related organ weight differences were observed, demonstrating recovery.

At terminal euthanasia, DCR-A1203-related microscopic findings were observed in the liver, lymph nodes (draining, mandibular, and mesenteric), and subcutaneous administration sites. In the liver, microscopic findings included vacuolated/granular Kupffer cells (minimal to mild) in animals administered ≥100 mg/kg/dose, hepatocellular hypertrophy (minimal) in males administered 300 mg/kg/dose, and single cell necrosis (minimal) in a female administered 300 mg/kg/dose. In the lymph nodes and/or at the subcutaneous administration sites, vacuolated/granular macrophages (minimal to mild) were observed in animals administered ≥30 mg/kg/dose. These findings are consistent with observations with other therapeutics using similar siRNA platforms reported in literature.

At recovery euthanasia, DCR-A1203-related microscopic findings were again observed in the liver, lymph nodes, and subcutaneous administration sites. In the liver, microscopic findings included vacuolated/granular Kupffer cells (minimal) in animals administered ≥100 mg/kg/dose and in the lymph nodes and/or at the subcutaneous administration sites, vacuolated/granular macrophages (minimal to mild) were observed in animals administered ≥100 mg/kg/dose. The lower incidence and/or severity of findings observed at the recovery euthanasia (as compared to the terminal euthanasia) demonstrated ongoing yet incomplete recovery.

Peak plasma DCR-A1203 concentrations were observed over a range from 1 to 12 hours post dose on Days 1 and 29. Following Cmax, DCR-A1203 concentrations generally decreased through 48 hours (last time point collected) in males and females on Day 1 and on Day 29. Plasma DCR-A1203 concentrations were quantifiable on Day 57 in recovery animals at 100 and 300 mg/kg/dose.

DCR-A1203 exposure, in terms of the area under the concentration time curve (AUC) from time 0 to 48 hours post dose and the maximum measured concentration of DCR-A1203 in plasma, increased with dose level. The AUC from time 0 to 48 hours post dose increased by approximately 18-fold on Day 1 and approximately 15-fold on Day 29 over the 10-fold increase in dose level, showing a greater than dose-proportional increase on both evaluation days. The maximum measured concentration of DCR-A1203 in plasma increased by approximately 9-fold on Day 1 and approximately 7-fold on Day 29, showing an approximate dose-proportional increase on both evaluation days. Overall, plasma exposure was approximately equivalent on Day 1 and Day 29, with no signs of accumulation. Mean half-life was determined over 0 to 48 hours post dose and ranged from 3.76 to 4.12 hours on Day 1 and from 4.01 to 5.21 hours on Day 29. The calculated half-life should be interpreted with caution as it was determined over 0 to 48 hours post dose and DCR-A1203 concentrations were quantifiable in plasma up to 28 days post dose (Day 57). DCR-A1203 exposure, in terms of AUC(0-48 hr) and Cmax, was similar (≤1.6-fold) between male and female monkeys on both evaluation days; therefore, data are presented for males and females combined.

TABLE 5
Summary of Toxicokinetics of DCR-A1203 in Plasma - Sexes Combined

| | | AUC | AUC | | | | | | | | |
|---|---|---|---|---|---|---|---|---|---|---|---|
| Dose | | (0-48 hr) | (0-48 hr)/ | Cmax | | tmax | | tlast | T1/2^{a} | ARAUC | ARCmax |
| (mg/kg) | Day | (ng*hr/mL) | D | (ng/mL) | Cmax/D | (hr) | | (hr) | (hr) | (RATIO) | (RATIO) |
| 30 | 1 | 78600 | 2620 | 7640 | 255 | 1.5 | (1-4) | 48 | 4.10†⁷ | NA | NA |
| 100 | | 454000 | 4540 | 29800 | 298 | 4 | (1-12) | 48 | 3.76 | NA | NA |
| 300 | | 1380000 | 4610 | 65400 | 218 | 8 | (2-12) | 48 | 4.12 | NA | NA |
| 30 | 29 | 101000 | 3370 | 9260 | 309 | 1.5 | (1-4) | 48 | 4.86†⁷ | 1.3 | 1.3 |
| 100 | | 383000 | 3830 | 23300 | 233 | 4 | (1-12) | 48 | 4.01 | 0.87 | 0.85 |
| 300 | | 1480000 | 4940 | 61200 | 204 | 8 | (4-12) | 48 | 5.21 | 1.1 | 0.96 |

N = 8 (30 mg/kg sexes combined) or N = 12 (100 and 300 mg/kg sexes combined) unless otherwise noted as †ⁿ, where ⁿ = number. Median (min-max) presented for tmax; range for tlast was (48 hr-48 hr) for all dose groups. NA = not applicable. ARAUC was calculated using AUC(0-48 hr); D = dose.
^{a}The calculated half-life should be interpreted with caution as it was determined over 0 to 48 hours post dose and DCR-A1203 concentrations were quantifiable in plasma up to 28 days post dose (Day 57).

DCR-A1203 concentrations were quantifiable in liver and kidney at all dose levels on Day 31 (48 hours post dose relative to Day 29); liver concentrations increased less-than-dose-proportionally, and kidney concentrations increased nearly dose-proportionally. DCR-A1203 concentrations were quantifiable in liver and kidney at both dose levels (100 and 300 mg/kg) on Day 57; liver concentrations increased nearly dose-proportionally (2-fold) over the 100 to 300 mg/kg dose level, and kidney concentrations increased nearly dose-proportionally (2-fold) in females between 100 and 300 mg/kg, but concentrations in males decreased between 100 and 300 mg/kg. DCR-A1203 concentrations were higher in liver than in kidney. Liver concentrations were 84% and 98% and kidney concentrations were 65% and 8% of the terminal necropsy level (Day 31) at recovery necropsy (Day 57) for the 100 and 300 mg/kg groups, respectively. Consequently, the liver-to-kidney ratios were generally higher on Day 57 compared to Day 31. DCR-A1203 concentrations were 8- to 38-fold higher in liver than in kidney at terminal necropsy (Day 31) and 79- to 94-fold higher in liver than in kidney at recovery necropsy (Day 57) in terms of mean ratios (Table 6).

TABLE 6
Tissue to Plasma Ratios for DCR-A1203 in
Male and Female Monkeys - Sexes Combined^{a}

| Dose | | Liver | Kidney | Plasma | Liver/Plasma | Kidney/Plasma | Liver/Kidney |
|---|---|---|---|---|---|---|---|
| (mg/kg) | Day | (ng/g) | (ng/g) | (ng/mL) | Ratio | Ratio | Ratio |
| 30 | 31 | 621000 | 41800 | 15.8 | 52000 | 3600 | 16 |
| 100 | | 1510000 | 114000 | 80.9 | 23000 | 1800 | 38 |
| 300 | | 2670000 | 357000 | 2360 | 5000 | 780 | 7.7 |
| 100 | 57 | 1270000 | 74400† | 24.7 | 60000 | 2300 | 79 |
| 300 | | 2600000 | 28600 | 51.8 | 53000 | 590 | 94 |

^{a}Plasma mean concentrations are for 48 hours post dose relative to Day 29, or Day 57 prior to euthanasia.
Note†
female kidney concentration was approximately 14-fold lower than male kidney concentration.

Evidence of DCR-A1203 activity in the liver was demonstrated by a reduction of ALDH2 mRNA by ≥85% in the main study groups administered 30, 100, and 300 mg/kg DCR-A1203 relative to controls. There was no apparent difference in ALDH2 mRNA expression between males and females. There was no recovery of ALDH2 reduction in livers in either the 100 or 300 mg/kg/dose recovery groups (recovery was not assessed at 30 mg/kg/dose). No DCR-A1203 activity was detected in any of the tested extrahepatic tissues (kidney, esophagus, and bone marrow) in either the main or recovery study groups.

In conclusion, administration of DCR-A1203 by subcutaneous injection once every 28 days for a total of 2 doses, to cynomolgus monkeys at levels of 30, 100, and 300 mg/kg/dose was well tolerated. DCR-A1203-related, non-adverse changes occurred in several clinical pathology parameters (≥100 mg/kg/dose) and in microscopic pathology of liver (≥100 mg/kg/dose) and of lymph nodes and dose administration sites (≥30 mg/kg/dose). All DCR-A1203-related changes in clinical pathology were reversible, and microscopic changes trended toward recovery. Under the conditions of this study, the no-observed-adverse-effect level (NOAEL) was determined to be 300 mg/kg/dose, with associated AUC(0-48 hr) of 1,480,000 ng*hr/mL and Cmax of 61,200 ng/mL (males and females combined, Day 29).

### Example 5: A Cardiovascular, Respiratory and Central Nervous System Assessment of DP11663P:DP16281G (DCR-A1203) Following Subcutaneous Injection Administration to Conscious, Radiotelemetry-Instrumented Cynomolgus Monkeys

The objective of this study was to assess the potential acute effects of subcutaneous injection of DCR-A1203, on respiratory parameters, arterial blood pressure, heart rate, body temperature, and lead II electrocardiogram (ECG) as well as effects on the gross behavioral, physiological, and neurological state of conscious radiotelemetry-instrumented male cynomolgus monkeys.

Animals received single subcutaneous doses of 0 (vehicle; 0.9% saline), 30, 100, or 300 mg/kg in an escalating dose design. The study design was as follows:

TABLE 7
Experimental Design

| | | Dose | | |
|---|---|---|---|---|
| | Dose Level | Concentration | Dose Volume | Number of |
| Test Material | (mg/kg) | (mg/mL)^{a} | (mL/kg)^{b} | Males^{c} |
| 1 | 0 | 0 | 1.6 | 4 |
| (Vehicle Control: 0.9% | | | | |
| Sodium Chloride for Injection) | | | | |
| 2 | 30 | 18.75 | 1.6 | 4 |
| (DCR-A1203) | | | | |
| 3 | 100 | 62.5 | 1.6 | 4 |
| (DCR-A1203) | | | | |
| 4 | 300 | 187.5 | 1.6 | 4 |
| (DCR-A1203) | | | | |

^{a}Formulation concentration was corrected for purity and moisture content with a correction factor of 1.14.
^{b}Based on the most recent body weight measurement.
^{c}The same 4 animals were used for each dosing occasion with 7 days between doses. Animals were dosed in an escalating dose design starting with the vehicle (0 mg/kg) treatment.

The following parameters and end points were evaluated in this study: clinical signs, qualitative food consumption, heart rate, arterial blood pressure (systolic, diastolic, and mean arterial pressure), pulse pressure, body temperature, and ECG waveforms (from which the ECG intervals PR, QRS, QT, and heart rate-corrected QT [QTcB and QTcL] were derived), respiratory parameters (respiratory rate, tidal volume, and minute volume), neurological examinations, and bioanalysis.

A single subcutaneous administration of DCR-A1203 in an escalating dose design to male cynomolgus monkeys at dose levels of 30, 100, and 300 mg/kg resulted in no DCR-A1203-related cardiovascular, respiratory, or neurologic effects.

Therefore, the no-observed-effect level (NOEL) was 300 mg/kg.

### CONCLUSIONS

A single subcutaneous administration of DCR-A1203 in an escalating dose design to male cynomolgus monkeys at dose levels of 30, 100, and 300 mg/kg resulted in no DCR-A1203-related cardiovascular, respiratory, or neurologic effects. Therefore, the no-observed-effect level (NOEL) was 300 mg/kg.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification, and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or one or more modified nucleotides and/or one or more modified internucleotide linkages and/or one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of all examples, or exemplary language (e.g., “such as”) provided herein, is intended merely to better illuminate the invention, and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize or be able to ascertain using no more than routine experimentation, many equivalents to the specific embodiments of the invention described herein. Such equivalents are intended to be encompassed by the following claims.

### REFERENCES

Hurley, T. D., Edenberg, H. J., Li, T.-K., “The pharmacogenomics of alcoholism,” In: Pharmacogenomics: The Search for Individualized Therapies, Weinheim, Germany: Wiley-VCH, pp. 417-41 (2002).
Guillot, A., Ren, T., Jourdan, T., Pawlosky, R. J., Han, E., Kim, S-J, Zhang, L., Koob, G. G., and Gao, B., “Targeting liver aldehyde dehydrogenase-2 prevents heavy but not moderate alcohol drinking,” PROC NATL ACAD SCI USA., 2019, 116(51):25974-981.
Nutt, D. J., “The role of the opioid system in alcohol dependence,” J PSYCHOPHARMACOLOGY, 2014, 28:8-22.
Plosker, G. L., “Acamprosate: A review of its use in alcohol dependence,” DRUGS, 2015, 75:1255-68.
Suh, J. J., Pettinati, H. M., Kampman, K. M., O'Brien, C. P., “The status of disulfiram: A half of a century later,” J CLIN PSYCHOPHARMACOL., 2006, 26, 290-302.
Jørgensen, C. H., Pedersen, B., Tonnesen, H., “The efficacy of disulfiram for the treatment of alcohol use disorder,” ALCOHOL CLIN EXP RES., 2011, 35, 1749-58.
Blanc, M., Daeppen, J. B., “Does disulfiram still have a role in alcoholism treatment?” REV. MED. SUISSE, 2005, 1, 1728-1730, 1732-1733.
Malcolm, R., Olive, M. F., Lechner, W., “The safety of disulfiram for the treatment of alcohol and cocaine dependence in randomized clinical trials: Guidance for clinical practice,” EXPERT OPIN. DRUG SAF., 2008, 7:459-72.
Chen, C. H., Ferreira, J. C., Gross, E. R., Mochly-Rosen, D. “Targeting aldehyde dehydrogenase 2: New therapeutic opportunities,” PHYSIOL. REV., 2014, 94:1-34.
Edenberg, H. J., McClintick, J. N., “Alcohol dehydrogenases, aldehyde dehydrogenases, and alcohol use disorders: A critical review,” ALCOHOL. CLIN. EXP. RES., 2018, 42:2281-97.

What is claimed is:

1. An oligonucleotide for reducing expression of ALDH2, the oligonucleotide comprising an antisense strand comprising an antisense strand having a sequence from 5′ to 3′ set forth as UAAACUGAGUUUCAUCCACCGG (SEQ ID NO: 1) and a sense strand having a sequence from 5′ to 3′ set forth as GGUGGAUGAAACUCAGUUUAGCAGCCGAAAGGCUGC (SEQ ID NO: 2); or a pharmaceutically acceptable salt thereof.

2. The oligonucleotide of claim 1, wherein all the nucleotides are modified.

3. The oligonucleotide of claim 2, wherein the nucleotide comprises a 2′-fluoro or 2′-O-methyl modification.

4. The oligonucleotide of claim 3, wherein the following positions are modified with a 2′-O-methyl: positions 1-7 and 12-36 of the sense strand and/or positions 1, 6, 8-13 and 15-22 of the antisense strand.

5. The oligonucleotide of claim 3, wherein the following positions are modified with a 2′-fluoro: positions 8-11 of the sense strand and/or positions 2-5, 7 and 14 of the antisense strand.

6. The oligonucleotide of claim 2, wherein the oligonucleotide comprises at least one modified internucleotide linkage.

7. The oligonucleotide of claim 6, wherein at least one modified internucleotide linkage is a phosphorothioate linkage.

8. The oligonucleotide of claim 6, wherein the oligonucleotide has a phosphorothioate linkage between one or more of: positions 1 and 2 of the sense strand, positions 1 and 2 of the antisense strand, positions 2 and 3 of the antisense strand, positions 3 and 4 of the antisense strand, positions 20 and 21 of the antisense strand, and positions 21 and 22 of the antisense strand.

9. A double stranded oligonucleotide comprising a sense strand:
5′mG-S-mG-mU-mG-mG-mA-mU-fG-fA-fA-fA-mC-mU-mC-mA-mG-mU-mU-mU-mA-mG-mC-mA-mG-mC-mC-mG-[ademA-GalNAc]-[ademA-GalNAc]-[ademA-GalNAc]-mG-mG-mC-mU-mG-mC 3′ (SEQ ID NO: 3), and
an antisense strand:
5′ [MePhosphonate-4O-mU]-S-fA-S-fA-S-fA-fC-mU-fG-mA-mG-mU-mU-mU-mC-fA-mU-mC-mC-mA-mC-mC-S-mG-S-mG 3′ (SEQ ID NO: 6); or a pharmaceutically acceptable salt thereof,
wherein:
“-” between nucleosides represent a phosphodiester internucleoside linkage;
“—S—” between nucleosides represent a phosphorothioate internucleoside linkage;
mA represents 2′-O-methyladenosine ribonucleoside;
mG represents 2′-O-methylguanosine ribonucleoside;
mC represents 2′-O-methylcytidine ribonucleoside;
mU represents 2′-O-methyluridine ribonucleoside;
fA represents 2′-fluoro-adenosine deoxyribonucleoside;
fG represents 2′-fluoro-guanosine deoxyribonucleoside;
fC represents 2′-fluoro-cytidine deoxyribonucleoside;
fU represents 2′-fluoro-uridine deoxyribonucleoside;
[ademA-GalNAc] represents 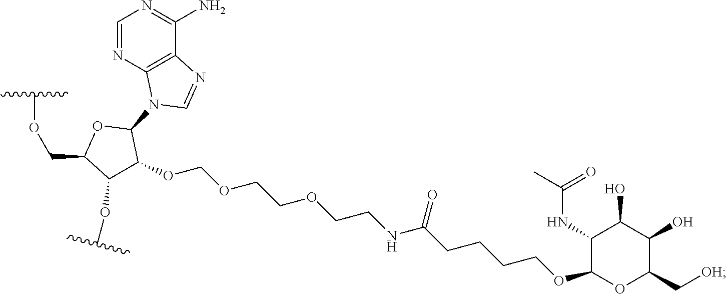 [SMILES: *OC[C@H]1O[C@@H](n2cnc3c(N)ncnc32)[C@H](OCOCCOCCNC(=O)CCCCO[C@@H]2O[C@H](CO)[C@H](O)[C@H](O)[C@H]2NC(C)=O)[C@@H]1O*]
and
[MePhosphonate-4O-mU] represents

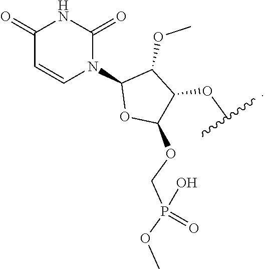 [SMILES: *O[C@@H]1[C@@H](OCP(=O)(O)OC)O[C@@H](n2ccc(=O)[nH]c2=O)[C@@H]1OC]

10. A sodium salt of a double stranded oligonucleotide comprising a sense strand:
5′ mG-S-mG-mU-mG-mG-mA-mU-fG-fA-fA-fA-mC-mU-mC-mA-mG-mU-mU-mU-mA-mG-mC-mA-mG-mC-mC-mG-[ademA-GalNAc]-[ademA-GalNAc]-[ademA-GalNAc]-mG-mG-mC-mU-mG-mC 3′ (SEQ ID NO: 3), and
an antisense strand:
5′ [MePhosphonate-4O-mU]-S-fA-S-fA-S-fA-fC-mU-fG-mA-mG-mU-mU-mU-mC-fA-mU-mC-mC-mA-mC-mC-S-mG-S-mG 3′ (SEQ ID NO: 6),
wherein:
“-” between nucleosides represent a phosphodiester internucleoside linkage;
“—S—” between nucleosides represent a phosphorothioate internucleoside linkage;
mA represents 2′-O-methyladenosine ribonucleoside;
mG represents 2′-O-methylguanosine ribonucleoside;
mC represents 2′-O-methylcytidine ribonucleoside;
mU represents 2′-O-methyluridine ribonucleoside;
fA represents 2′-fluoro-adenosine deoxyribonucleoside;
fG represents 2′-fluoro-guanosine deoxyribonucleoside;
fC represents 2′-fluoro-cytidine deoxyribonucleoside;
fU represents 2′-fluoro-uridine deoxyribonucleoside;
[ademA-GalNAc] represents 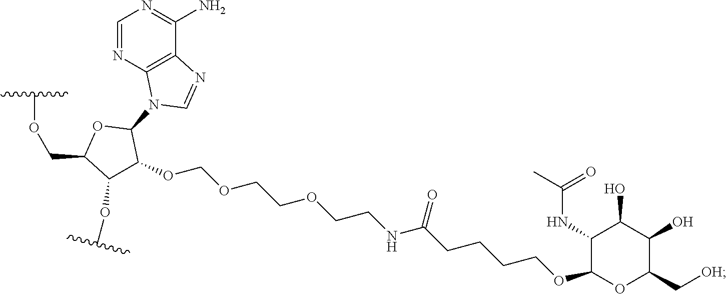 [SMILES: *OC[C@H]1O[C@@H](n2cnc3c(N)ncnc32)[C@H](OCOCCOCCNC(=O)CCCCO[C@@H]2O[C@H](CO)[C@H](O)[C@H](O)[C@H]2NC(C)=O)[C@@H]1O*]
and
[MePhosphonate-4O-mU] represents

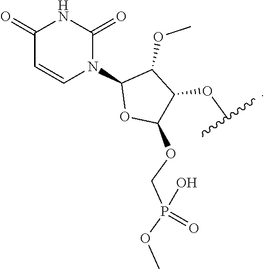 [SMILES: *O[C@@H]1[C@@H](OCP(=O)(O)OC)O[C@@H](n2ccc(=O)[nH]c2=O)[C@@H]1OC]

11. A composition comprising a double stranded oligonucleotide, wherein the double stranded oligonucleotide comprises a sense strand:
5′ mG-S-mG-mU-mG-mG-mA-mU-fG-fA-fA-fA-mC-mU-mC-mA-mG-mU-mU-mU-mA-mG-mC-mA-mG-mC-mC-mG-[ademA-GalNAc]-[ademA-GalNAc]-[ademA-GalNAc]-mG-mG-mC-mU-mG-mC 3′(SEQ ID NO: 3), and
an antisense strand:
5′ [MePhosphonate-4O-mU]-S-fA-S-fA-S-fA-fC-mU-fG-mA-mG-mU-mU-mU-mC-fA-mU-mC-mC-mA-mC-mC-S-mG-S-mG 3′ (SEQ ID NO: 6); or a pharmaceutically acceptable salt thereof,
wherein:
“-” between nucleosides represent a phosphodiester internucleoside linkage;
“—S—” between nucleosides represent a phosphorothioate internucleoside linkage;
mA represents 2′-O-methyladenosine ribonucleoside;
mG represents 2′-O-methylguanosine ribonucleoside;
mC represents 2′-O-methylcytidine ribonucleoside;
mU represents 2′-O-methyluridine ribonucleoside;
fA represents 2′-fluoro-adenosine deoxyribonucleoside;
fG represents 2′-fluoro-guanosine deoxyribonucleoside;
fC represents 2′-fluoro-cytidine deoxyribonucleoside;
fU represents 2′-fluoro-uridine deoxyribonucleoside;
[ademA-GalNAc] represents 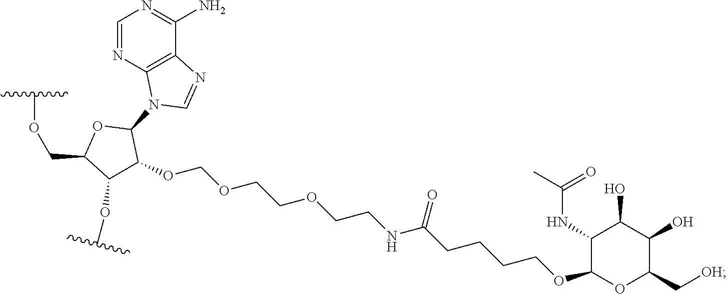 [SMILES: *OC[C@H]1O[C@@H](n2cnc3c(N)ncnc32)[C@H](OCOCCOCCNC(=O)CCCCO[C@@H]2O[C@H](CO)[C@H](O)[C@H](O)[C@H]2NC(C)=O)[C@@H]1O*]
and
[MePhosphonate-4O-mU] represents

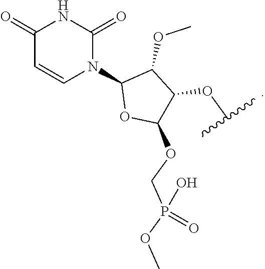 [SMILES: *O[C@@H]1[C@@H](OCP(=O)(O)OC)O[C@@H](n2ccc(=O)[nH]c2=O)[C@@H]1OC]

12. The composition of claim 11, further comprising a pharmaceutically acceptable carrier, excipient, or adjuvant.

13. The composition of claim 11 or 12, wherein the pharmaceutically acceptable salt is a sodium salt of the oligonucleotide.

14. A method of delivering an oligonucleotide to a subject, the method comprising administering the oligonucleotide of any one or claims 1-10 or the composition of any one of claims 11-13 to the subject.

15. A method of decreasing ethanol tolerance in a subject, the method comprising administering the oligonucleotide of any one of claims 1-10 or the composition of any one of claims 11-13 to the subject.

16. A method of inhibiting ethanol intake by a subject, the method comprising administering the oligonucleotide of any one of claim 10 or the composition of any one of claims 11-13 to the subject.

17. A method of decreasing the ability of a subject to metabolize ethanol, the method comprising administering the oligonucleotide of any one of claims 1-10 or the composition any one of claims 11-13 to the subject.

18. The method of claim 17, wherein the subject suffers from alcoholism.

19. A method of reducing the levels of ADH1B, ADH1C, and ALDH2 in a subject, the method comprising administering the oligonucleotide of any one of claims 1-10 or the composition any one of claims 11-13 to the subject.

20. A method of reducing the levels of ALDH2 in a subject, the method comprising administering the oligonucleotide of any one of claims 1-10 or the composition any one of claims 11-13 to the subject.

21. A method of treating a subject suffering from alcoholism, the method comprising administering the oligonucleotide of any one of claims 1-10 or the composition any one of claims 11-13 in combination with another ALDH2 inhibitor.

22. The method of claim 21, wherein the other ALDH2 inhibitor is disulfiram.


SEQUENCE LISTING (9 sequences)

SEQ ID NO: 1 - DNA, 22 bp, Artificial Sequence
  Synthetic sequence
uaaacugaguuucauccaccgg

SEQ ID NO: 2 - DNA, 36 bp, Artificial Sequence
  Synthetic sequence
gguggaugaaacucaguuuagcagccgaaaggcugc

SEQ ID NO: 3 - DNA, 36 bp, Artificial Sequence; identical to SEQ ID NO: 2
  Synthetic sequence
  modified_base (1)..(1): 2′-O-methylguanosine ribonucleotide
  misc_feature (1)..(2): modified by phosphorothioate internucleotide linkage
  modified_base (2)..(2): 2′-O-methylguanosine ribonucleotide
  modified_base (3)..(3): 2′-O-methyluridine ribonucleotide
  modified_base (4)..(4): 2′-O-methylguanosine ribonucleotide
  modified_base (5)..(5): 2′-O-methylguanosine ribonucleotide
  modified_base (6)..(6): 2′-O-methyladenosine ribonucleotide
  modified_base (7)..(7): 2′-O-methyluridine ribonucleotide
  modified_base (8)..(8): 2′-fluoro-guanosine deoxyribonucleotide
  modified_base (9)..(9): 2′-fluoro-adenosine deoxyribonucleotide
  modified_base (10)..(10): 2′-fluoro-adenosine deoxyribonucleotide
  modified_base (11)..(11): 2′-fluoro-adenosine deoxyribonucleotide
  modified_base (12)..(12): 2′-O-methylcytidine ribonucleotide
  modified_base (13)..(13): 2′-O-methyluridine ribonucleotide
  modified_base (14)..(14): 2′-O-methylcytidine ribonucleotide
  modified_base (15)..(15): 2′-O-methyladenosine ribonucleotide
  modified_base (16)..(16): 2′-O-methylguanosine ribonucleotide
  modified_base (17)..(17): 2′-O-methyluridine ribonucleotide
  modified_base (18)..(18): 2′-O-methyluridine ribonucleotide
  modified_base (19)..(19): 2′-O-methyluridine ribonucleotide
  modified_base (20)..(20): 2′-O-methyladenosine ribonucleotide
  modified_base (21)..(21): 2′-O-methylguanosine ribonucleotide
  modified_base (22)..(22): 2′-O-methylcytidine ribonucleotide
  modified_base (23)..(23): 2′-O-methyladenosine ribonucleotide
  modified_base (24)..(24): 2′-O-methylguanosine ribonucleotide
  modified_base (25)..(25): 2′-O-methylcytidine ribonucleotide
  modified_base (26)..(26): 2′-O-methylcytidine ribonucleotide
  modified_base (27)..(27): 2′-O-methylguanosine ribonucleotide
  modified_base (28)..(28): ademA-GalNAc
  modified_base (29)..(29): ademA-GalNAc
  modified_base (30)..(30): ademA-GalNAc
  modified_base (31)..(31): 2′-O-methylguanosine ribonucleotide
  modified_base (32)..(32): 2′-O-methylguanosine ribonucleotide
  modified_base (33)..(33): 2′-O-methylcytidine ribonucleotide
  modified_base (34)..(34): 2′-O-methyluridine ribonucleotide
  modified_base (35)..(35): 2′-O-methylguanosine ribonucleotide
  modified_base (36)..(36): 2′-O-methylcytidine ribonucleotide

SEQ ID NO: 4 - DNA, 22 bp, Artificial Sequence
  Synthetic sequence
  modified_base (1)..(1): 5′-methoxy, phosphonate-4′-oxy-2′-O- methyluridine
  misc_feature (1)..(4): modified by phosphorothioate internucleotide linkage
  modified_base (2)..(2): 2′-fluoro-adenosine deoxyribonucleotide
  modified_base (3)..(3): 2′-fluoro-adenosine deoxyribonucleotide
  modified_base (4)..(4): 2′-fluoro-adenosine deoxyribonucleotide
  modified_base (5)..(5): 2′-O-methylcytidine ribonucleotide
  modified_base (6)..(6): 2′-O-methyluridine ribonucleotide
  modified_base (7)..(7): 2′-fluoro-guanosine deoxyribonucleotide
  modified_base (8)..(8): 2′-O-methyladenosine ribonucleotide
  modified_base (9)..(9): 2′-O-methylguanosine ribonucleotide
  modified_base (10)..(10): 2′-O-methyluridine ribonucleotide
  modified_base (11)..(11): 2′-O-methyluridine ribonucleotide
  modified_base (12)..(12): 2′-O-methyluridine ribonucleotide
  modified_base (13)..(13): 2′-O-methylcytidine ribonucleotide
  modified_base (14)..(14): 2′-fluoro-adenosine deoxyribonucleotide
  modified_base (15)..(15): 2′-O-methyluridine ribonucleotide
  modified_base (16)..(16): 2′-O-methylcytidine ribonucleotide
  modified_base (17)..(17): 2′-O-methylcytidine ribonucleotide
  modified_base (18)..(18): 2′-O-methyladenosine ribonucleotide
  modified_base (19)..(19): 2′-O-methylcytidine ribonucleotide
  modified_base (20)..(20): 2′-O-methylcytidine ribonucleotide
  misc_feature (20)..(22): modified by phosphorothioate internucleotide linkage
  modified_base (21)..(21): 2′-O-methylguanosine ribonucleotide
  modified_base (22)..(22): 2′-O-methylguanosine ribonucleotide
uaaacugaguuucauccaccgg

SEQ ID NO: 5 - DNA, 22 bp, Artificial Sequence
  Synthetic sequence
  modified_base (1)..(1): 5′-methoxy, phosphonate-4′-oxy-2′-O- methyluridine
  misc_feature (1)..(4): modified by phosphorothioate internucleotide linkage
  modified_base (2)..(2): 2′-fluoro-adenosine deoxyribonucleotide
  modified_base (3)..(3): 2′-fluoro-adenosine deoxyribonucleotide
  modified_base (4)..(4): 2′-fluoro-adenosine deoxyribonucleotide
  modified_base (5)..(5): 2′-O-methylcytidine ribonucleotide
  modified_base (6)..(6): 2′-O-methyluridine ribonucleotide
  modified_base (7)..(7): 2′-fluoro-guanosine deoxyribonucleotide
  modified_base (8)..(8): 2′-O-methyladenosine ribonucleotide
  modified_base (9)..(9): 2′-O-methylguanosine ribonucleotide
  modified_base (10)..(10): 2′-fluoro-uridine deoxyribonucleotide
  modified_base (11)..(11): 2′-fluoro-uridine deoxyribonucleotide
  modified_base (12)..(12): 2′-fluoro-uridine deoxyribonucleotide
  modified_base (13)..(13): 2′-O-methylcytidine ribonucleotide
  modified_base (14)..(14): 2′-fluoro-adenosine deoxyribonucleotide
  modified_base (15)..(15): 2′-O-methyluridine ribonucleotide
  modified_base (16)..(16): 2′-O-methylcytidine ribonucleotide
  modified_base (17)..(17): 2′-O-methylcytidine ribonucleotide
  modified_base (18)..(18): 2′-O-methyladenosine ribonucleotide
  modified_base (19)..(19): 2′-O-methylcytidine ribonucleotide
  modified_base (20)..(20): 2′-O-methylcytidine ribonucleotide
  misc_feature (20)..(22): modified by phosphorothioate internucleotide linkage
  modified_base (21)..(21): 2′-O-methylguanosine ribonucleotide
  modified_base (22)..(22): 2′-O-methylguanosine ribonucleotide
uaaacugaguuucauccaccgg

SEQ ID NO: 6 - DNA, 22 bp, Artificial Sequence
  Artificial sequence
  modified_base (1)..(1): 5′-methoxy, phosphonate-4′-oxy-2′-O- methyluridine
  misc_feature (1)..(4): modified by phosphorothioate internucleotide linkage
  modified_base (2)..(2): 2′-fluoro-adenosine deoxyribonucleotide
  modified_base (3)..(3): 2′-fluoro-adenosine deoxyribonucleotide
  modified_base (4)..(4): 2′-fluoro-adenosine deoxyribonucleotide
  modified_base (5)..(5): 2′-fluoro-cytidine deoxyribonucleotide
  modified_base (6)..(6): 2′-O-methyluridine ribonucleotide
  modified_base (7)..(7): 2′-fluoro-guanosine deoxyribonucleotide
  modified_base (8)..(8): 2′-O-methyladenosine ribonucleotide
  modified_base (9)..(9): 2′-O-methylguanosine ribonucleotide
  modified_base (10)..(10): 2′-O-methyluridine ribonucleotide
  modified_base (11)..(11): 2′-O-methyluridine ribonucleotide
  modified_base (12)..(12): 2′-O-methyluridine ribonucleotide
  modified_base (13)..(13): 2′-O-methylcytidine ribonucleotide
  modified_base (14)..(14): 2′-fluoro-adenosine deoxyribonucleotide
  modified_base (15)..(15): 2′-O-methyluridine ribonucleotide
  modified_base (16)..(16): 2′-O-methylcytidine ribonucleotide
  modified_base (17)..(17): 2′-O-methylcytidine ribonucleotide
  modified_base (18)..(18): 2′-O-methyladenosine ribonucleotide
  modified_base (19)..(19): 2′-O-methylcytidine ribonucleotide
  modified_base (20)..(20): 2′-O-methylcytidine ribonucleotide
  misc_feature (20)..(22): modified by phosphorothioate internucleotide linkage
  modified_base (21)..(21): 2′-O-methylguanosine ribonucleotide
  modified_base (22)..(22): 2′-O-methylguanosine ribonucleotide
uaaacugaguuucauccaccgg

SEQ ID NO: 7 - DNA, 22 bp, Artificial Sequence
  Synthetic sequence
  modified_base (1)..(1): 5′-methoxy, phosphonate-4′-oxy-2′-O- methyluridine
  misc_feature (1)..(4): modified by phosphorothioate internucleotide linkage
  modified_base (2)..(2): 2′-fluoro-adenosine deoxyribonucleotide
  modified_base (3)..(3): 2′-fluoro-adenosine deoxyribonucleotide
  modified_base (4)..(4): 2′-fluoro-adenosine deoxyribonucleotide
  modified_base (5)..(5): 2′-fluoro-cytidine deoxyribonucleotide
  modified_base (6)..(6): 2′-O-methyluridine ribonucleotide
  modified_base (7)..(7): 2′-fluoro-guanosine deoxyribonucleotide
  modified_base (8)..(8): 2′-O-methyladenosine ribonucleotide
  modified_base (9)..(9): 2′-O-methylguanosine ribonucleotide
  modified_base (10)..(10): 2′-fluoro-uridine deoxyribonucleotide
  modified_base (11)..(11): 2′-O-methyluridine ribonucleotide
  modified_base (12)..(12): 2′-O-methyluridine ribonucleotide
  modified_base (13)..(13): 2′-O-methylcytidine ribonucleotide
  modified_base (14)..(14): 2′-fluoro-adenosine deoxyribonucleotide
  modified_base (15)..(15): 2′-O-methyluridine ribonucleoside
  modified_base (16)..(16): 2′-O-methylcytidine ribonucleotide
  modified_base (17)..(17): 2′-O-methylcytidine ribonucleotide
  modified_base (18)..(18): 2′-O-methyladenosine ribonucleotide
  modified_base (19)..(19): 2′-O-methylcytidine ribonucleotide
  modified_base (20)..(20): 2′-O-methylcytidine ribonucleotide
  misc_feature (20)..(22): modified by phosphorothioate internucleotide linkage
  modified_base (21)..(21): 2′-O-methylguanosine ribonucleotide
  modified_base (22)..(22): 2′-O-methylguanosine ribonucleotide
uaaacugaguuucauccaccgg

SEQ ID NO: 8 - DNA, 36 bp, Artificial Sequence; identical to SEQ ID NO: 2
  Synthetic sequence
  modified_base (1)..(1): 2′-O-methylguanosine ribonucleotide
  misc_feature (1)..(2): modified by phosphorothioate internucleotide linkage
  modified_base (2)..(2): 2′-O-methylguanosine ribonucleotide
  modified_base (3)..(3): 2′-fluoro-uridine deoxyribonucleotide
  modified_base (4)..(4): 2′-O-methylguanosine ribonucleotide
  modified_base (5)..(5): 2′-fluoro-guanosine deoxyribonucleotide
  modified_base (6)..(6): 2′-O-methyladenosine ribonucleotide
  modified_base (7)..(7): 2′-O-methyluridine ribonucleotide
  modified_base (8)..(8): 2′-fluoro-guanosine deoxyribonucleotide
  modified_base (9)..(9): 2′-O-methyladenosine ribonucleotide
  modified_base (10)..(10): 2′-fluoro-adenosine deoxyribonucleotide
  modified_base (11)..(11): 2′-O-methyladenosine ribonucleotide
  modified_base (12)..(12): 2′-fluoro-guanosine deoxyribonucleotide
  modified_base (13)..(13): 2′-O-methyluridine ribonucleotide
  modified_base (14)..(14): 2′-O-methylcytidine ribonucleotide
  modified_base (15)..(15): 2′-fluoro-adenosine deoxyribonucleotide
  modified_base (16)..(16): 2′-O-methylguanosine ribonucleotide
  modified_base (17)..(17): 2′-fluoro-uridine deoxyribonucleotide
  modified_base (18)..(18): 2′-O-methyluridine ribonucleotide
  modified_base (19)..(19): 2′-O-methyluridine ribonucleotide
  modified_base (20)..(20): 2′-O-methyladenosine ribonucleotide
  modified_base (21)..(21): 2′-O-methylguanosine ribonucleotide
  modified_base (22)..(22): 2′-O-methylcytidine ribonucleotide
  modified_base (23)..(23): 2′-O-methyladenosine ribonucleotide
  modified_base (24)..(24): 2′-O-methylguanosine ribonucleotide
  modified_base (25)..(25): 2′-O-methylcytidine ribonucleotide
  modified_base (26)..(26): 2′-O-methylcytidine ribonucleotide
  modified_base (27)..(27): 2′-O-methylguanosine ribonucleotide
  modified_base (28)..(28): ademA-GalNAc
  modified_base (29)..(29): ademA-GalNAc
  modified_base (30)..(30): ademA-GalNAc
  modified_base (31)..(31): 2′-O-methylguanosine ribonucleotide
  modified_base (32)..(32): 2′-O-methylguanosine ribonucleotide
  modified_base (33)..(33): 2′-O-methylcytidine ribonucleotide
  modified_base (34)..(34): 2′-O-methyluridine ribonucleotide
  modified_base (35)..(35): 2′-O-methylguanosine ribonucleotide
  modified_base (36)..(36): 2′-O-methylcytidine ribonucleotide

SEQ ID NO: 9 - DNA, 22 bp, Artificial Sequence
  Synthetic sequence
  modified_base (1)..(1): 5′-methoxy, phosphonate-4′-oxy-2′-O- methyluridine
  misc_feature (1)..(3): modified by phosphorothioate internucleotide linkage
  modified_base (2)..(2): 2′-fluoro-adenosine deoxyribonucleotide
  modified_base (3)..(3): 2′-fluoro-adenosine deoxyribonucleotide
  modified_base (4)..(4): 2′-fluoro-adenosine deoxyribonucleotide
  modified_base (5)..(5): 2′-fluoro-cytidine deoxyribonucleotide
  modified_base (6)..(6): 2′-O-methyluridine ribonucleotide
  modified_base (7)..(7): 2′-fluoro-guanosine deoxyribonucleotide
  modified_base (8)..(8): 2′-O-methyladenosine ribonucleotide
  modified_base (9)..(9): 2′-O-methylguanosine ribonucleotide
  modified_base (10)..(10): 2′-fluoro-uridine deoxyribonucleotide
  modified_base (11)..(11): 2′-O-methyluridine ribonucleotide
  modified_base (12)..(12): 2′-O-methyluridine ribonucleotide
  modified_base (13)..(13): 2′-O-methylcytidine ribonucleotide
  modified_base (14)..(14): 2′-fluoro-adenosine deoxyribonucleotide
  modified_base (15)..(15): 2′-O-methyluridine ribonucleotide
  modified_base (16)..(16): 2′-fluoro-cytidine deoxyribonucleotide
  modified_base (17)..(17): 2′-O-methylcytidine ribonucleotide
  modified_base (18)..(18): 2′-O-methyladenosine ribonucleotide
  modified_base (19)..(19): 2′-fluoro-cytidine deoxyribonucleotide
  modified_base (20)..(20): 2′-fluoro-cytidine deoxyribonucleotide
  misc_feature (20)..(22): modified by phosphorothioate internucleotide linkage
  modified_base (21)..(21): 2′-O-methylguanosine ribonucleotide
  modified_base (22)..(22): 2′-O-methylguanosine ribonucleotide
uaaacugaguuucauccaccgg